(19) United States
(12) Patent Application Publication
(10) Pub. No.: US 2023/0017301 A1
(43) Pub. Date: Jan. 19, 2023

(54) SKIRT ASSEMBLY FOR IMPLANTABLE PROSTHETIC VALVE

(71) Applicant: Edwards Lifesciences Corporation, Irvine, CA (US)

(72) Inventors: Elena Sherman, Pardes Hana (IL); Michael Bukin, Pardes Hana (IL); Sandip Vasant Pawar, Irvine, CA (US); Shahram Zamani, Newport Coast, CA (US); Tamir S. Levi, Zikhron Yaakov (IL); Noam Nir, Pardes-Hanna (IL); Yana Mayatskaya, Or-Aqiva (IL); Alexander Barash, Tzoran (IL); Ziv Yohanan, Kfar Hahoresh (IL); Nikolay Gurovich, Hadera (IL); Itai Pelled, Ramat-Hasharon (IL)

(21) Appl. No.: 17/935,891

(22) Filed: Sep. 27, 2022

Related U.S. Application Data
(63) Continuation of application No. PCT/US2021/024777, filed on Mar. 30, 2021.
(60) Provisional application No. 63/003,773, filed on Apr. 1, 2020.

(51) Int. Cl. A61F 2/24

(52) U.S. Cl. CPC A61F 2/2418; A61F 2/2415; A61F 2220/0075; A61F 2210/0076

(57) ABSTRACT

An implantable prosthetic device can include a frame movable between a radially compressed and a radially expanded configuration and a sealing member. The frame can include an inflow end portion, an outflow end portion, and a plurality of struts. The sealing member can be disposed around the frame and can include a cushioning layer comprising a plurality of texturized yarns extending along the longitudinal axis of the frame, and a base layer disposed between the cushioning layer and the frame.

### CROSS REFERENCE TO RELATED APPLICATION

This is a continuation of PCT patent application no. PCT/US2021/024777, filed on Mar. 30, 2021, which application claims the benefit of U.S. Provisional Application Ser. No. 63/003,773, entitled SKIRT ASSEMBLY FOR IMPLANTABLE PROSTHETIC VALVE, filed on Apr. 1, 2020, which application is incorporated by reference herein in its entirety.

### FIELD

The present disclosure concerns embodiments of a prosthetic valve for implantation into body ducts, such as native heart valve annuluses.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Prosthetic heart valves that can expand without the use of a mechanical actuator or balloon can be referred to as “self-expanding”prosthetic heart valves. Each type of prosthetic heart valve can include various advantages.

In a procedure to implant a prosthetic heart valve, the prosthetic heart valve is typically positioned in the annulus of a native heart valve and expanded or allowed to expand to its functional size. In order to retain the prosthetic heart valve at the desired location, the prosthetic heart valve may be larger than the diameter of the native valve annulus such that it applies force to the surrounding tissue in order to prevent the prosthetic heart valve from becoming dislodged. Over time, relative motion of the prosthetic heart valve and tissue of the native heart valve (e.g., native valve leaflets, chordae tendineae, etc.) in contact with the prosthetic heart valve may cause damage to the tissue. Accordingly, there is a need for improvements to prosthetic heart valves.

### SUMMARY

In a representative embodiment, an implantable prosthetic device can comprise a frame movable between a radially compressed and a radially expanded configuration, the frame comprising an inflow end portion, an outflow end portion, and a plurality of struts, and a sealing member. The sealing member can be disposed around the frame and can include a cushioning layer comprising a plurality of texturized yarns extending along the longitudinal axis of the frame and a base layer disposed between the cushioning layer and the frame.

In another representative embodiment, an implantable prosthetic valve can comprise a frame movable between a radially compressed and a radially expanded configuration, the frame comprising an inflow end portion, an outflow end portion, and a plurality of struts, and a sealing member. The sealing member can circumscribe the frame and can comprise a first layer disposed radially outwardly of the frame, the first layer configured to promote radially outward thrombus formation between the implantable prosthetic device and a selected implantation site, and a second layer disposed between the first layer and the frame, the second layer configured to inhibit radially inward thrombus formation.

In a representative embodiment, a method of making an angled weave fabric can comprise disposing a first set of yarns on a loom such that the yarns extend in a first direction, weaving a shuttle coupled to a yarn through the first set of yarns in a second direction such that a portion of the yarn is oriented perpendicularly relative to the first set of yarns, and moving the portion of the yarn against an angled base member such that the portion is oriented at a non-perpendicular angle relative to the first set of yarns.

In a representative embodiment, a method of making an implantable prosthetic device can comprise expanding a mechanically-expandable frame comprising a first set of struts and a second set of struts, wherein each strut of the first set of struts is coupled to one or more struts of the second set of struts, to a non-working diameter wherein the first set of struts are oriented perpendicularly with respect to the second set of struts, disposing a sealing member comprising a plurality of warp and weft yarns over the frame such that the warp yarns are aligned with the first set of struts and the weft yarns are aligned with the second set of struts, and coupling the sealing member to the frame.

In a representative embodiment, an implantable prosthetic device comprises a frame movable between a radially compressed and a radially expanded configuration, the frame comprising an inflow end portion, an outflow end portion, and a plurality of struts defining a plurality of cells, and a sealing member. The sealing member comprises at least one expandable suture configured to move between a default configuration and a swollen configuration when immersed in blood.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of a prosthetic heart valve, according to one embodiment.

FIG. 2A is a side elevation view of the frame of the prosthetic heart valve of FIG. 1, shown in a radially compressed state.

FIG. 2B is a side elevation view of the frame of the prosthetic heart valve of FIG. 1, shown in a radially expanded state.

FIG. 3 is a perspective view of a prosthetic valve frame, shown in a radially collapsed state, having a plurality of expansion and locking mechanisms, according to another embodiment.

FIG. 4 is a perspective view of the frame and the expansion and locking mechanisms of FIG. 3, with the frame shown in a radially expanded state.

FIG. 5A is a perspective view of a screw of one of the expansion and locking mechanisms of FIG. 3.

FIG. 5B is a perspective view of one of the expansion and locking mechanisms of FIG. 3.

FIG. 5C is another perspective view of the frame and the expansion and locking mechanisms of FIG. 3, with the frame shown in a radially expanded state.

FIG. 6 is another perspective view of one of the expansion and locking mechanisms of FIG. 3.

FIG. 7 shows a cross sectional view of one of the expansion and locking mechanisms of FIG. 3 along with a portion of the frame.

FIG. 8 is side elevation view of a delivery apparatus for a prosthetic heart valve, according to one embodiment.

FIG. 9A is a perspective view of a prosthetic heart valve comprising a sealing member, according to one embodiment.

FIG. 9B is a perspective view of a prosthetic heart valve comprising a sealing member, according to another embodiment.

FIG. 10 is a perspective view of the base layer of the sealing member of FIGS. 9A-9B.

FIG. 11 is a perspective view of the cushioning layer of the sealing member of FIGS. 9A-9B.

FIG. 12 is a magnified plan view of the sealing member of FIG. 9A prior to being cut out.

FIG. 13 is a magnified plan view of another embodiment of the sealing member of FIG. 9A prior to being cut out.

FIG. 14 is a perspective view of the inflow end portion of the frame and sealing member of the prosthetic valve of FIG. 9B.

FIG. 15 illustrates an exemplary weave pattern for use in a sealing member, according to one embodiment.

FIG. 16 is a plan view of an exemplary loom for use in creating an exemplary weave pattern for use in a sealing member, according to one embodiment.

FIG. 17 is a magnified plan view of a sealing member of prior to being cut out, according to one embodiment.

FIG. 18 is a perspective view of the prosthetic heart valve of FIG. 9B.

FIG. 19A is a plan view of a portion of the sealing member of FIG. 18 including multiple leno weave portions.

FIG. 19B is a magnified view of a portion of the sealing member of FIG. 18 including a leno weave portion.

FIG. 20 illustrates an exemplary leno weave pattern, according to one embodiment.

FIG. 21 is a perspective view of the prosthetic heart valve of FIG. 18, including a leno weave portion.

FIG. 22 is a plan view of a portion of the sealing member of FIGS. 9A-9B including multiple exemplary elongated yarns.

FIG. 23 is a side elevation view of a portion of the sealing member of FIGS. 9A-9B including an exemplary chain stitch.

FIGS. 24A-24C illustrate an exemplary chain stitching technique.

FIGS. 25A-25D illustrate a schematic embodiment of a prosthetic heart valve in various states of expansion and compression, according to one embodiment.

FIGS. 26A-26C illustrate a magnified portion of a woven sealing member in various states of expansion and compression, according to one embodiment.

FIG. 27 illustrates a portion of the sealing member of FIGS. 26A-26C overlaid on a portion of the prosthetic heart valve of FIGS. 25A-25D.

FIG. 28 is a side elevation view of the prosthetic heart valve of FIGS. 25A-25D shown in the fully compressed configuration.

FIG. 29 is a side elevation view of the prosthetic heart valve of FIGS. 25A-25D shown in a partially expanded non-working configuration.

FIG. 30 is a side elevation view of a portion of the prosthetic heart valve of FIGS. 25A-25D shown in the fully expanded configuration.

FIG. 31 is a side elevation view of a portion of a sealing member coupled to a strut, according to one embodiment.

FIG. 32 is a perspective view of a frame for a prosthetic heart valve including a sealing member, according to one embodiment.

FIG. 33A is a perspective view of an expandable suture in the tensioned configuration.

FIG. 33B is a perspective view of the expandable suture of FIG. 33A in the relaxed or puffy configuration.

FIG. 34 is a perspective view of a prosthetic heart valve, according to one embodiment.

FIGS. 35-38 are perspective view of various exemplary sealing members comprising expandable sutures.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein. For example, the sealing member 410 of prosthetic valve 400 can be used with prosthetic valves 10, 100, 302, 700, 1000, 1100, etc. and the expandable sutures of prosthetic valves 1000 and 1100 can be used with prosthetic valves 10, 100, 302, 400, 700, etc.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively, with respect to a prosthetic valve implanted at the aortic position (within the native aortic valve). Thus, for example, the lower end of the valve is its inflow end and the upper end of the valve is its outflow end for a prosthetic aortic valve. As used herein, the term “proximal” refers to a position, direction, or portion of a device that is closer to the user (the physician operating the delivery device for implanting the implant) or a handle of a delivery device and further away from the implantation site. As used herein, the term “distal” refers to a position, direction, or portion of a device that is further away from the user and the handle and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses, and examples thereof will be discussed in more detail later.

In various embodiments described herein, delivery apparatuses, prosthetic valves, and methods may be deployed or performed within a subject. Subjects include (but are not limited to) medical patients, veterinary patients, animal models, cadavers, and simulators of the cardiac and vasculature system (e.g., anthropomorphic phantoms and explant tissue). Accordingly, various embodiments are directed to devices and/or methods for medical procedures, practice of medical procedures, and/or training of medical procedures. Simulators may include a simulation of whole or partial vasculature system, a whole or partial heart, and/or whole or partial components of the vasculature system (e.g., whole or partial ascending aorta). References to native tissue (e.g., native heart valve) refer to preexisting structures within the subject, such as (for example) native tissue of a patient or a component of a simulator.

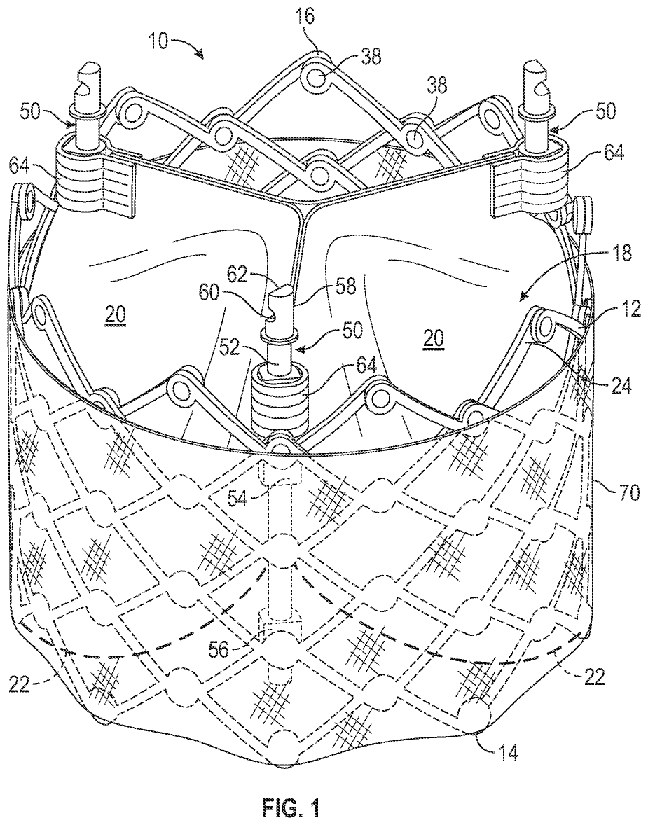

FIG. 1 shows an exemplary prosthetic valve 10, according to one embodiment. Any of the prosthetic valves disclosed herein are adapted to be implanted in the native aortic annulus, although in other embodiments they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve).

The prosthetic valve 10 can include an annular stent or frame 12 having an inflow end 14 and an outflow end 16. The prosthetic valve 10 can also include a valvular structure 18 which is coupled to and supported inside of the frame 12. The valvular structure 18 is configured to regulate the flow of blood through the prosthetic valve 10 from the inflow end 14 to the outflow end 16.

The valvular structure 18 can include, for example, a leaflet assembly comprising one or more leaflets 20 made of a flexible material. The leaflets 20 can be made from in whole or part, biological material, bio-compatible synthetic materials, or other such materials. Suitable biological material can include, for example, bovine pericardium (or pericardium from other sources). The leaflets 20 can be secured to one another at their adjacent sides to form commissures, each of which can be secured to a respective actuator 50 or the frame 102.

In the depicted embodiment, the valvular structure 18 comprises three leaflets 20, which can be arranged to collapse in a tricuspid arrangement. Each leaflet 20 can have an inflow edge portion 22. As shown in FIG. 1, the inflow edge portions 22 of the leaflets 20 can define an undulating, curved scallop shape that follows or tracks a plurality of interconnected strut segments of the frame 12 in a circumferential direction when the frame 12 is in the radially expanded configuration. The inflow edges of the leaflets can be referred to as a “scallop line.”

In some embodiments, the inflow edge portions 22 of the leaflets 20 can be sutured to adjacent struts of the frame generally along the scallop line. In other embodiments, the inflow edge portions 22 of the leaflets 20 can be sutured to an inner skirt, which in turn in sutured to adjacent struts of the frame. By forming the leaflets 20 with this scallop geometry, stresses on the leaflets 20 are reduced, which in turn improves durability of the valve 10. Moreover, by virtue of the scallop shape, folds and ripples at the belly of each leaflet 20 (the central region of each leaflet), which can cause early calcification in those areas, can be eliminated or at least minimized. The scallop geometry also reduces the amount of tissue material used to form valvular structure 18, thereby allowing a smaller, more even crimped profile at the inflow end 14 of the valve 10.

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, and 8,252,202, U.S. Publication Nos. 2018/0325665 and 2020/0352711, all of which are incorporated herein by reference in their entireties.

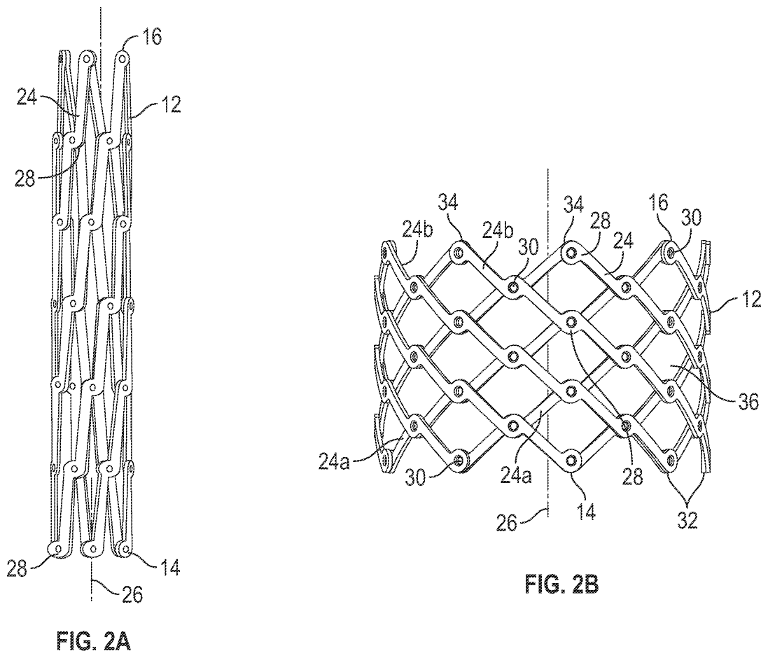

The prosthetic valve 10 can be radially compressible and expandable between a radially compressed configuration and a radially expanded configuration. FIGS. 2A-2B show the bare frame 12 of the prosthetic valve 10 (without the leaflets and other components) for purposes of illustrating expansion of the prosthetic valve 10 from the radially compressed configuration (FIG. 2A) to the radially expanded configuration (FIG. 2B).

The frame 12 can include a plurality of interconnected lattice struts 24 arranged in a lattice-type pattern and forming a plurality of apices 34 at the outflow end 16 of the prosthetic valve 10. The struts 24 can also form similar apices 32 at the inflow end 14 of the prosthetic valve 10. In FIG. 2B, the struts 24 are shown as positioned diagonally, or offset at an angle relative to, and radially offset from, a longitudinal axis 26 of the prosthetic valve 10 when the prosthetic valve 10 is in the expanded configuration. In other implementations, the struts 24 can be offset by a different amount than depicted in FIG. 2B, or some or all of the struts 24 can be positioned parallel to the longitudinal axis 26 of the prosthetic valve 10.

The struts 24 can comprise a set of inner struts 24a (extending from the lower left to the upper right of the frame in FIG. 2B) and a set of outer struts 24b (extending from the upper left to the lower right of the frame in FIG. 2B) connected to the inner struts 24a. The open lattice structure of the frame 12 can define a plurality of open frame cells 36 between the struts 24.

The struts 24 can be pivotably coupled to one another at one or more pivot joints or pivot junctions 28 along the length of each strut. For example, in one embodiment, each of the struts 24 can be formed with apertures 30 at opposing ends of the strut and apertures spaced along the length of the strut. Respective hinges can be formed at the locations where struts 24 overlap each other via fasteners 38 (FIG. 1), such as rivets or pins that extend through the apertures 30. The hinges can allow the struts 24 to pivot relative to one another as the frame 12 is radially expanded or compressed, such as during assembly, preparation, or implantation of the prosthetic valve 10.

The frame struts and the components used to form the pivot joints of the frame 12 (or any frames described below) can be made of any of various suitable materials, such as stainless steel, a cobalt chromium alloy, or a nickel titanium alloy (“NiTi”), for example Nitinol. In some embodiments, the frame 12 can be constructed by forming individual components (e.g., the struts and fasteners of the frame) and then mechanically assembling and connecting the individual components together. Further details regarding the construction of the frame and the prosthetic valve are described in U.S. Pat. Nos. 10,603,165 and 10,869,759 and U.S. Patent Publication Nos. 2019/0060057 and 2020/0188099, all of which are incorporated herein by reference.

In the illustrated embodiment, the prosthetic valve 10 can be mechanically expanded from the radially contracted configuration to the radially expanded configuration. For example, the prosthetic valve 10 can be radially expanded by maintaining the inflow end 14 of the frame 12 at a fixed position while applying a force in the axial direction against the outflow end 16 toward the inflow end 14. Alternatively, the prosthetic valve 10 can be expanded by applying an axial force against the inflow end 14 while maintaining the outflow end 16 at a fixed position, or by applying opposing axial forces to the inflow and outflow ends 14, 16, respectively.

As shown in FIG. 1, the prosthetic valve 10 can include one or more actuators 50 mounted to and equally spaced around the inner surface of the frame 12. Each of the actuators 50 can be configured to form a releasable connection with one or more respective actuators of a delivery apparatus.

In the illustrated embodiment, expansion and compression forces can be applied to the frame by the actuators 50. Referring again to FIG. 1, each of the actuators 50 can comprise a screw or threaded rod 52, a first anchor in the form of a cylinder or sleeve 54, and a second anchor in the form of a threaded nut 56. The rod 52 extends through the sleeve 54 and the nut 56. The sleeve 54 can be secured to the frame 12, such as with a fastener 38 that forms a hinge at the junction between two struts. Each actuator 50 is configured to increase the distance between the attachment locations of a respective sleeve 54 and nut 56, which causes the frame 12 to elongate axially and compress radially, and to decrease the distance between the attachment locations of a respective sleeve 54 and nut 56, which causes the frame 12 to foreshorten axially and expand radially.

For example, each rod 52 can have external threads that engage internal threads of the nut 56 such that rotation of the rod causes corresponding axial movement of the nut 56 toward or away from the sleeve 54 (depending on the direction of rotation of the rod 52). This causes the hinges supporting the sleeve 54 and the nut 56 to move closer towards each other to radially expand the frame or to move farther away from each other to radially compress the frame, depending on the direction of rotation of the rod 52.

In other embodiments, the actuators 50 can be reciprocating type actuators configured to apply axial directed forces to the frame to produce radial expansion and compression of the frame. For example, the rod 52 of each actuator can be fixed axially relative to the sleeve 56 and slidable relative to the sleeve 54. Thus, in this manner, moving the rod 52 distally relative to the sleeve 54 and/or moving the sleeve 54 proximally relative to the rod 52 radially compresses the frame. Conversely, moving the rod 52 proximally relative to the sleeve 54 and/or moving the sleeve 54 distally relative to the rod 52 radially expands the frame.

When reciprocating type actuators are used, the prosthetic valve can also include one or more locking mechanisms that retain the frame in the expanded state. The locking mechanisms can be separate components that are mounted on the frame apart from the actuators, or they can be a sub-component of the actuators themselves.

Each rod 52 can include an attachment member 58 along a proximal end portion of the rod 52 configured to form a releasable connection with a corresponding actuator of a delivery apparatus. The actuator(s) of the delivery apparatus can apply forces to the rods for radially compressing or expanding the prosthetic valve 10. The attachment member 58 in the illustrated configuration comprises a notch 60 and a projection 62 that can engage a corresponding projection of an actuator of the delivery apparatus.

In the illustrated embodiments, the prosthetic valve 10 includes three such actuators 50, although a greater or fewer number of actuators could be used in other embodiments. The leaflets 20 can have commissure attachments members 64 that wrap around the sleeves 54 of the actuators 50. Further details of the actuators, locking mechanisms and delivery apparatuses for actuating the actuators can be found in U.S. Pat. No. 10,603,165 and U.S. Patent Publication Nos. 2019/0060057, and 2018/0325665, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

The prosthetic valve 10 can include one or more skirts or sealing members. In some embodiments, the prosthetic valve 10 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. As shown in FIG. 1, the prosthetic valve 10 can also include an outer skirt 70 mounted on the outer surface of the frame 12. The outer skirt 70 can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Patent Publication No. 2020/0352711, which is incorporated herein by reference in its entirety.

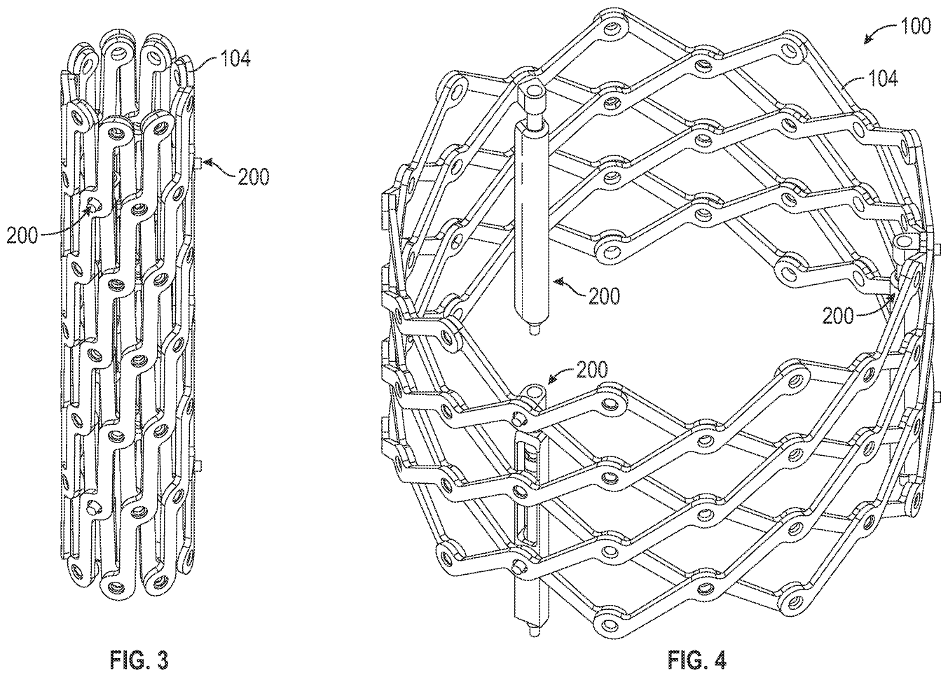

FIGS. 3-4 show another embodiment of a prosthetic valve 100 comprising a frame 104 and expansion and locking mechanisms 200 (also referred to as “actuators”). It should be understood that the prosthetic valve 100 can include leaflets 20 and other soft components, such as one or more skirts 70, which are removed for purposes of illustration. Expansion and locking mechanism 200 can be used to both radially expand and lock the prosthetic valve in a radially expanded state. In the example of FIGS. 3 and 4, three expansion and locking mechanisms 200 are attached to the frame 104 but in other example delivery assemblies, any number of expansion and locking mechanisms 200 can be used. FIG. 3 shows the expansion and locking mechanisms 200 attached to the frame 104 when the frame is in a radially collapsed configuration and FIG. 4 shows expansion and locking mechanisms attached to the frame when the frame is in a radially expanded configuration.

It will be appreciated that prosthetic valve 100 can, in certain embodiments, use other mechanisms for expansion and locking, such as linear actuators, alternate locking mechanisms, and alternate expansion and locking mechanisms. Further details regarding the use of linear actuators, locking mechanisms, and expansion and locking mechanisms in prosthetic valve can be found, for example, in U.S. Patent Publication No. 10,603,165, which is incorporated herein by reference in its entirety.

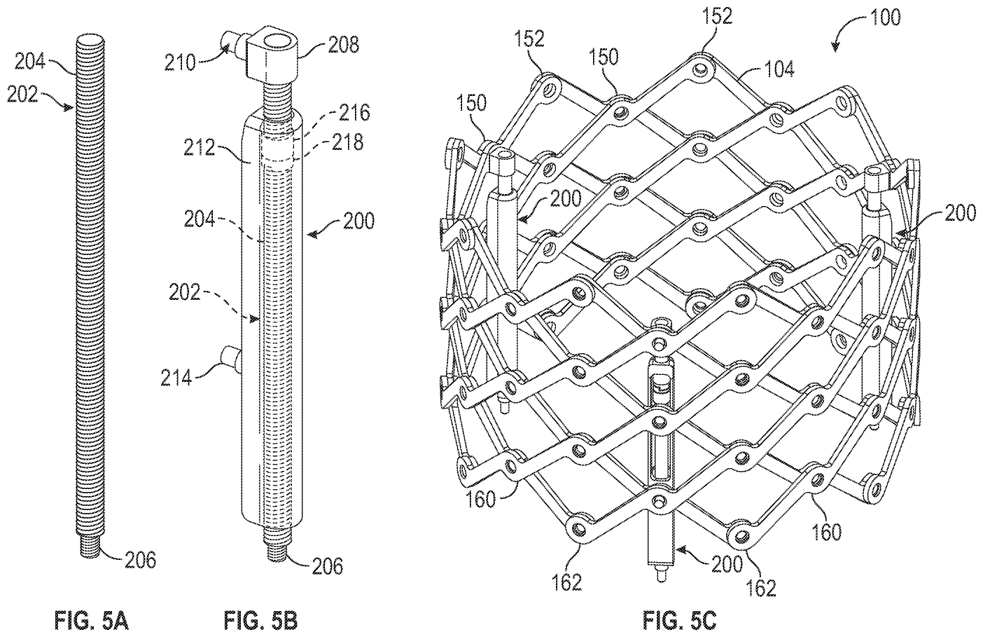

Referring to FIGS. 5A-5C, the expansion and locking mechanism 200 in the illustrated embodiment can include an actuator screw 202 (which functions as a linear actuator or a push-pull member in the illustrated embodiment) comprising a relatively long upper, or distal, portion 204 and a relatively shorter lower, or proximal, portion 206 at the proximal end of the screw 200, wherein the lower portion has a smaller diameter than the upper portion. Both the upper and lower portions 204, 206 of the screw 202 can have externally threaded surfaces.

The actuator screw 200 can have a distal attachment piece 208 attached to its distal end having a radially extending distal valve connector 210. The distal attachment piece 208 can be fixed to the screw 202 (e.g., welded together or manufactured as one piece). The distal valve connector 210 can extend through an opening at or near the distal end of the frame 104 formed at a location on the frame where two or more struts intersect as shown in FIG. 5C. The distal valve connector 210 can be fixed to the frame 104 (e.g., welded). Due to the shape of the struts, the distal end of the frame 104 comprises an alternating series of distal junctions 150 and distal apices 152. In the illustrated example, the distal valve connectors 210 of the three expansion and locking mechanisms 200 are connected to the frame 104 through distal junctions 150. In other examples, one or more distal valve connectors 210 can be connected to the frame 104 through distal apices 152. In other embodiments, the distal valve connectors 210 can be connected to junctions closer to the proximal end of the frame 104.

The expansion and locking mechanism 200 can further include a sleeve 212. The sleeve 212 can be positioned annularly around the distal portion 204 of the screw 202 and can contain axial openings at its proximal and distal ends through which the screw 202 can extend. The axial openings and the lumen in the sleeve 212 can have a diameter larger than the diameter of the distal portion 204 of the screw 202 such that the screw can move freely within the sleeve (the screw 202 can be moved proximally and distally relative to the sleeve 212). Because the actuator screw 202 can move freely within the sleeve, it can be used to radially expand and/or contract the frame 104 as disclosed in further detail below.

The sleeve 212 can have a proximal valve connector 214 extending radially from its outer surface. The proximal valve connector 214 can be fixed to the sleeve 212 (e.g., welded). The proximal valve connector 214 can be axially spaced from the distal valve connector 210 such that the proximal valve connector can extend through an opening at or near the proximal end of the frame 104. The proximal end of the frame 104 comprises an alternating series of proximal junctions 160 and proximal apices 162. In the illustrated example, the proximal valve connectors 214 of the three expansion and locking mechanisms 200 are connected to the frame 104 through proximal junctions 160. In other examples, one or more proximal valve connectors 214 can be connected to the frame 104 through proximal apices 162. In other embodiments, the proximal valve connectors 214 can be connected to junctions closer to the distal end of the frame 104.

It should be understood that the distal and proximal connectors 210, 214 need not be connected to opposite ends of the frame. The actuator 200 can be used to expand and compress the frame as long as the distal and proximal connectors are connected to respective junctions on the frame that are axially spaced from each other.

A locking nut 216 can be positioned inside of the sleeve 212 and can have an internally threaded surface that can engage the externally threaded surface of the actuator screw 202. The locking nut 216 can have a notched portion 218 at its proximal end, the purpose of which is described below. The locking nut can be used to lock the frame 104 into a particularly radially expanded state, as discussed below.

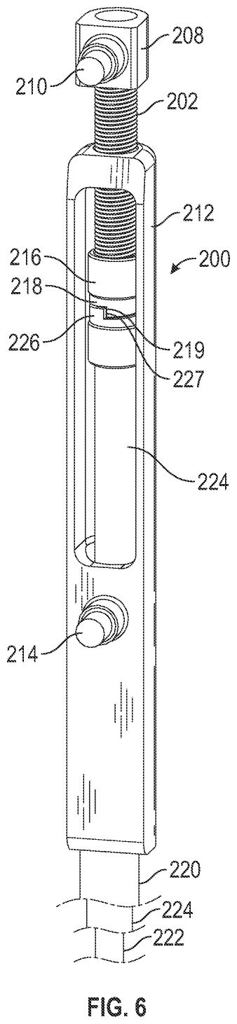

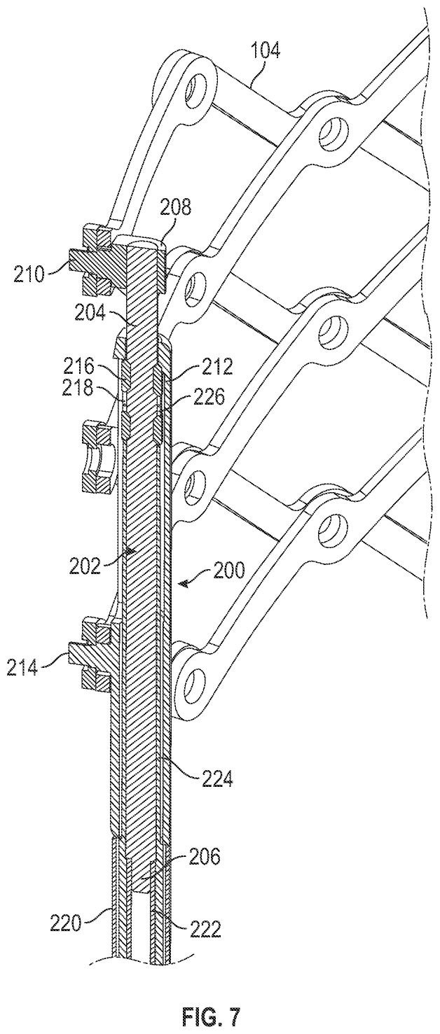

FIGS. 6 and 7 shows the expansion and locking mechanism 200 including components of a delivery apparatus not shown in FIGS. 5A-5C. As shown, the expansion and locking mechanism 200 can be releasably coupled to a support tube 220, an actuator member 222, and a locking tool 224. The proximal end of the support tube 220 can be connected to a handle or other control device (not shown) that a doctor or operator of the delivery assembly utilizes to operate the expansion and locking mechanism 200 as described herein. Similarly, the proximal ends of the actuator member 222 and the locking tool 224 can be connected to the handle.

The support tube 220 annularly surrounds a proximal portion of the locking tool 224 such that the locking tool extends through a lumen of the support tube. The support tube 220 and the sleeve are sized such that the distal end of the support tube abuts or engages the proximal end of the sleeve 212 such that the support tube is prevented from moving distally beyond the sleeve.

The actuator member 222 extends through a lumen of the locking tool 224. The actuator member 222 can be, for example, a shaft, a rod, a cable, or wire. The distal end portion of the actuator member 222 can be releasably connected to the proximal end portion 206 of the actuator screw 202. For example, the distal end portion of the actuator member 222 can have an internally threaded surface that can engage the external threads of the proximal end portion 206 of the actuator screw 202. Alternatively, the actuator member 222 can have external threads that engage an internally threaded portion of the screw 202. When the actuator member 222 is threaded onto the actuator screw 202, axial movement of the actuator member causes axial movement of the screw.

The distal portion of the locking tool 224 annularly surrounds the actuator screw 202 and extends through a lumen of the sleeve 212 and the proximal portion of the locking tool annularly surrounds the actuator member 222 and extends through a lumen of the support tube 220 to the handle of the delivery device. The locking tool 224 can have an internally threaded surface that can engage the externally threaded surface of the locking screw 202 such that clockwise or counter-clockwise rotation of the locking tool 224 causes the locking tool to advance distally or proximally along the screw, respectively.

The distal end of the locking tool 224 can comprise a notched portion 226, as can best be seen in FIG. 6. The notched portion 226 of the locking tool 224 can have an engagement surface 227 that is configured to engage a correspondingly shaped engagement surface 219 of the notched portion 218 of the locking nut 216 such that rotation of the locking tool (e.g., clockwise rotation) causes the nut 216 to rotate in the same direction (e.g., clockwise) and advance distally along the locking screw 202. The notched portions 218, 226 in the illustrated embodiment are configured such that rotation of the locking tool 224 in the opposite direction (e.g., counter-clockwise) allows the notched portion 226 of the tool 224 to disengage the notched portion 218 of the locking nut 216; that is, rotation of the locking tool in a direction that causes the locking tool to move proximally does not cause corresponding rotation of the nut.

In alternative embodiments, the distal end portion of the locking tool 224 can have various other configurations adapted to engage the nut 216 and produce rotation of the nut upon rotation of the locking tool for moving the nut distally, such as any of the tool configurations described herein. In some embodiments, the distal end portion of the locking tool 224 can be adapted to produce rotation of the nut 216 in both directions so as move the nut distally and proximally along the locking screw 202.

In operation, prior to implantation, the actuator member 222 is screwed onto the proximal end portion 206 of the actuator screw 202 and the locking nut 216 is rotated such that it is positioned at the proximal end of the screw. The frame 104 can then be placed in a radially collapsed state and the delivery assembly can be inserted into a subject. Once the prosthetic valve is at a desired implantation site, the frame 104 can be radially expanded as described herein.

To radially expand the frame 104, the support tube 220 is held firmly against the sleeve 212. The actuator member 222 is then pulled in a proximal direction through the support tube, such as by pulling on the proximal end of the actuator member or actuating a control knob on the handle that produces proximal movement of the actuator member. Because the support tube 220 is being held against the sleeve 212, which is connected to a proximal end of the frame 104 by the proximal valve connector 214, the proximal end of the frame is prevented from moving relative to the support tube. As such, movement of the actuator member 222 in a proximal direction causes movement of the actuator screw 202 in a proximal direction (because the actuator member is threaded onto the screw), thereby causing the frame 104 to foreshorten axially and expand radially. Alternatively, the frame 104 can be expanded by moving the support tube 220 distally while holding the actuator member 222 stationary or moving the support tube distally while moving the actuator member 222 proximally.

After the frame 104 is expanded to a desired radially expanded size, the frame can be locked at this radially expanded size as described herein. Locking the frame can be achieved by rotating the locking tool 224 in a clockwise direction causing the notched portion 226 of the locking tool to engage the notched portion 218 of the locking nut 216, thereby advancing the locking nut distally along the actuator screw 202. The locking tool 224 can be so rotated until the locking nut 216 abuts an internal shoulder at the distal end of the sleeve 212 and the locking nut 216 cannot advance distally any further (see FIG. 6). This will prevent the screw 202 from advancing distally relative to the sleeve 212 and radially compressing the frame 104. However, in the illustrated embodiment, the nut 216 and the screw 202 can still move proximally through the sleeve 212, thereby allowing additional expansion of the frame 104 either during implantation or later during a valve-in-valve procedure.

Once the frame 104 is locked in radially expanded state, the locking tool 224 can be rotated in a direction to move the locking tool proximally (e.g., in a counter-clockwise direction) to decouple the notched portion 226 from the notched portion 218 of the locking nut 216 and to unscrew the locking tool from the actuator screw 202. Additionally, the actuator member 222 can be rotated in a direction to unscrew the actuator member from the lower portion 206 of the actuator screw 202 (e.g., the actuator member 222 can be configured to disengage from the actuator screw when rotated counter-clockwise). Once the locking tool 224 and the actuator member 222 are unscrewed from the actuator screw 202, they can be removed from the subject along with the support tube 220, leaving the actuator screw and the sleeve 212 connected to the frame 104, as shown in FIG. 5C, with the frame 104 locked in a particular radially-expanded state.

In an alternative embodiment, the locking tool 224 can be formed without internal threads that engage the external threads of the actuator screw 202, which can allow the locking tool 224 to be slid distally and proximally through the sleeve 212 and along the actuator screw 202 to engage and disengage the nut 216.

In some embodiments, additional designs for expansion and locking mechanisms can be used instead of the design previously described. Details on expansion and locking mechanisms can be found, for example, in U.S. Patent Application Publication No. 10,603,165, which is incorporated herein by reference in its entirety.

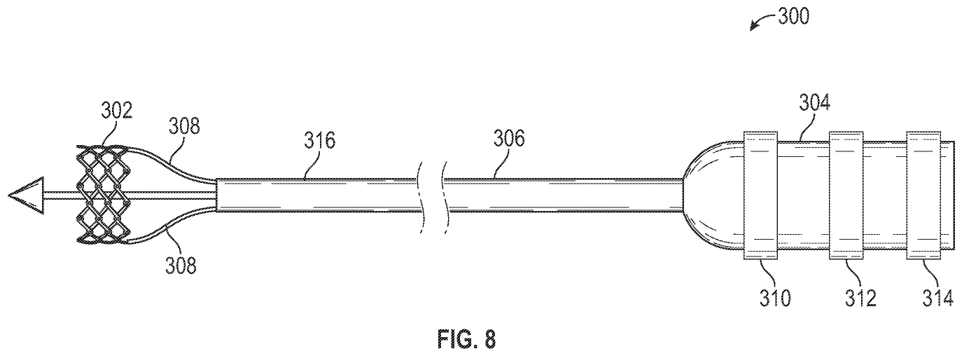

FIG. 8 illustrates a delivery apparatus 300, according to one embodiment, adapted to deliver a prosthetic heart valve 302, such as the illustrated prosthetic heart valve 10 or 100, described above. The prosthetic valve 302 can be releasably coupled to the delivery apparatus 300. It should be understood that the delivery apparatus 300 and other delivery apparatuses disclosed herein can be used to implant prosthetic devices other than prosthetic valves, such as stents or grafts.

The delivery apparatus 300 in the illustrated embodiment generally includes a handle 304, a first elongated shaft 306 (which comprises an outer shaft in the illustrated embodiment) extending distally from the handle 304, at least one actuator assembly 308 extending distally through the outer shaft 306. The at least one actuator assembly 308 can be configured to radially expand and/or radially collapse the prosthetic valve 302 when actuated.

Though the illustrated embodiment shows two actuator assemblies 308 for purposes of illustration, it should be understood that one actuator 308 can be provided for each actuator on the prosthetic valve. For example, three actuator assemblies 308 can be provided for a prosthetic valve having three actuators. In other embodiments, a greater or fewer number of actuator assemblies can be present.

In some embodiments, a distal end portion of the shaft 306 can be sized to house the prosthetic valve in its radially compressed, delivery state during delivery of the prosthetic valve through a vasculature. In this manner, the distal end portion functions as a delivery sheath 316 or capsule for the prosthetic valve during delivery,

The actuator assemblies 308 can be releasably coupled to the prosthetic valve 302. For example, in the illustrated embodiment, each actuator assembly 308 can be coupled to a respective actuator 200 of the prosthetic valve 302. Each actuator assembly 308 can comprise a support tube 220, an actuator member 222, and a locking tool 224. When actuated, the actuator assembly can transmit pushing and/or pulling forces to portions of the prosthetic valve to radially expand and collapse the prosthetic valve as previously described. The actuator assemblies 308 can be at least partially disposed radially within, and extend axially through, one or more lumens of the outer shaft 306. For example, the actuator assemblies 308 can extend through a central lumen of the shaft 306 or through separate respective lumens formed in the shaft 306.

The handle 304 of the delivery apparatus 300 can include one or more control mechanisms (e.g., knobs or other actuating mechanisms) for controlling different components of the delivery apparatus 300 in order to expand and/or deploy the prosthetic valve 10. For example, in the illustrated embodiment the handle 304 comprises first, second, and third knobs 310, 312, and 314.

The first knob 310 can be a rotatable knob configured to produce axial movement of the outer shaft 306 relative to the prosthetic valve 302 in the distal and/or proximal directions in order to deploy the prosthetic valve from the delivery sheath 316 once the prosthetic valve has been advanced to a location at or adjacent the desired implantation location with the subject. For example, rotation of the first knob 310 in a first direction (e.g., clockwise) can retract the sheath 316 proximally relative to the prosthetic valve 302 and rotation of the first knob 310 in a second direction (e.g., counter-clockwise) can advance the sheath 316 distally. In other embodiments, the first knob 310 can be actuated by sliding or moving the knob 310 axially, such as pulling and/or pushing the knob. In other embodiments, actuation of the first knob 310 (rotation or sliding movement of the knob 310) can produce axial movement of the actuator assemblies 308 (and therefore the prosthetic valve 302) relative to the delivery sheath 316 to advance the prosthetic valve distally from the sheath 316.

The second knob 312 can be a rotatable knob configured to produce radial expansion and/or contraction of the prosthetic valve 302. For example, rotation of the second knob 312 can move the actuator member 222 and the support tube 220 axially relative to one another. Rotation of the second knob 312 in a first direction (e.g., clockwise) can radially expand the prosthetic valve 302 and rotation of the second knob 312 in a second direction (e.g., counter-clockwise) can radially collapse the prosthetic valve 302. In other embodiments, the second knob 312 can be actuated by sliding or moving the knob 312 axially, such as pulling and/or pushing the knob.

The third knob 314 can be a rotatable knob configured to retain the prosthetic heart valve 302 in its expanded configuration. For example, the third knob 314 can be operatively connected to a proximal end portion of the locking tool 224 of each actuator assembly 308. Rotation of the third knob in a first direction (e.g., clockwise) can rotate each locking tool 224 to advance the locking nuts 216 to their distal positions to resist radial compression of the frame of the prosthetic valve, as described above. Rotation of the knob 314 in the opposite direction (e.g., counterclockwise) can rotate each locking tool 224 in the opposite direction to decouple each locking tool 224 from the respective nut 216 and remove the locking tool 224 from the respective actuator screw 202. In other embodiments, the third knob 314 can be actuated by sliding or moving the third knob 314 axially, such as pulling and/or pushing the knob.

Although not shown, the handle 304 can include a fourth rotatable knob operative connected to a proximal end portion of each actuator member 222. The fourth knob can be configured to rotate each actuator member 222, upon rotation of the knob, to unscrew each actuator member 222 from the proximal portion 206 of a respective actuator 202. As described above, once the locking tools 224 and the actuator members 222 are unscrewed form the actuator screws 202, they can be removed from the subject along with the support tubes 220.

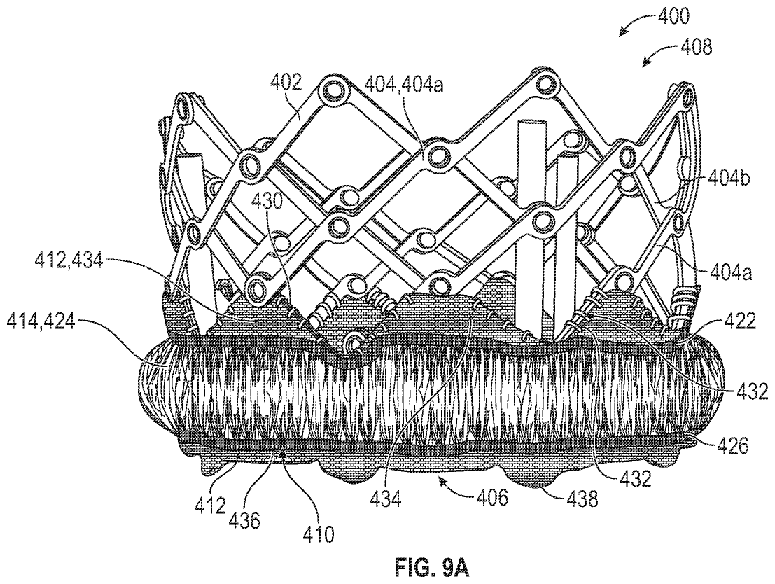

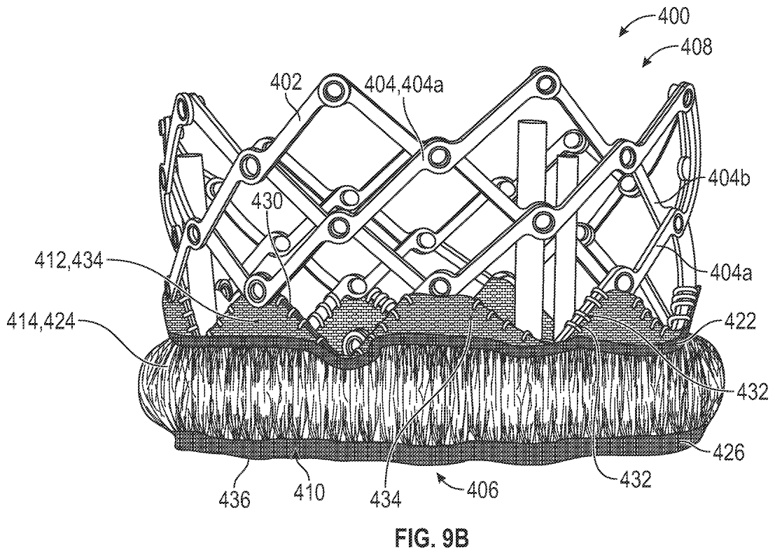

FIGS. 9-11 illustrate a representative embodiment of a prosthetic heart valve 400 comprising a frame 402. The prosthetic heart valve 400 can include a valvular structure (e.g., valvular structure 18), and actuators (e.g., actuators 50) as previously described, though these components are omitted for purposes of illustration. The frame 402 can comprise a plurality of interconnected struts 404 which extend from the inflow end portion 406 to the outflow end portion 408 of the frame 402. The plurality of struts 404 can comprise radially disposed outer or first struts 404a and radially disposed inner or second struts 404b. In the illustrated embodiment, the frame 402 is a mechanically expandable frame, however, in other embodiments, the frame can be a self-expanding frame, a balloon-expandable frame, or a hybrid mechanically-expandable and self-expanding frame.

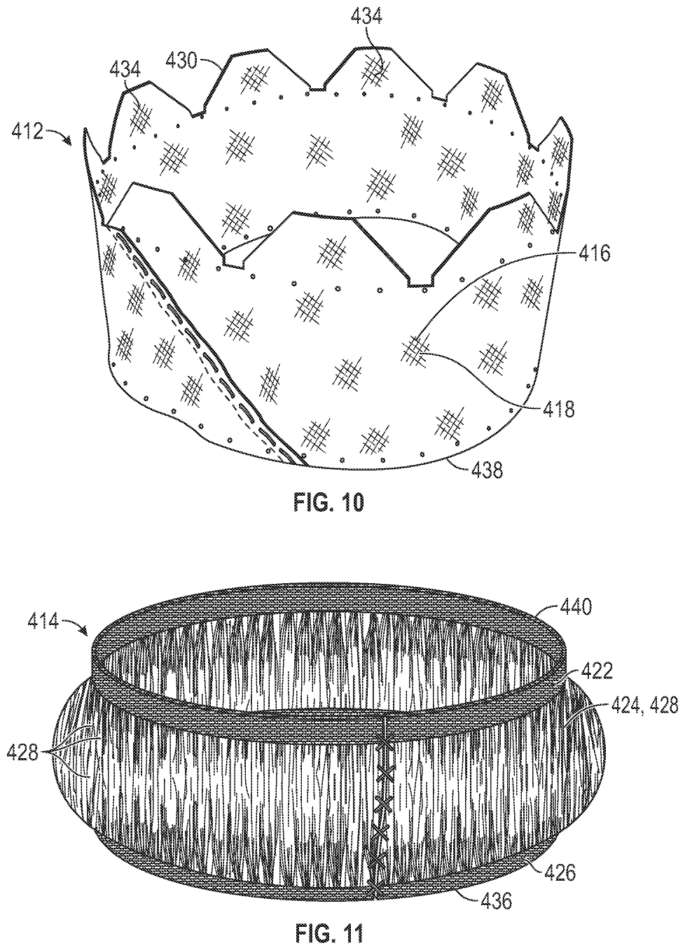

The prosthetic valve 400 can further comprise a sealing member 410. The sealing member 410 can comprise an inner, base layer 412 (FIG. 10) that can be disposed on the radially facing outer surface of the frame 402 and/or on the radially facing inner surface of the frame 402, and an outer, cushioning layer 414, configured, for example, as a cloth layer (FIG. 11). As shown in FIG. 10, the base layer 412 can be a woven layer comprising a plain weave pattern having a first set of yarns 416 extending in a first direction and configured as warp yarns and second set of yarns 418 extending in a second direction and configured as weft yarns. As used herein, the term “yarn” can refer to a yarn having a plurality of filaments or a monofilament yarn.

In the illustrated embodiment, the sealing member 410 comprises a base layer 412 and a cushioning layer 414 disposed around and attached directly to the base layer 412, such as with stitching, as further described below. A pre-assembled sealing member 410 (comprising the base layer 412 and the cushioning layer 414) can be mounted on the outside of a frame 402 of a prosthetic valve, as shown in FIG. 9. Various methods for forming the sealing member and mounting it to a frame of a prosthetic valve are described in detail below.

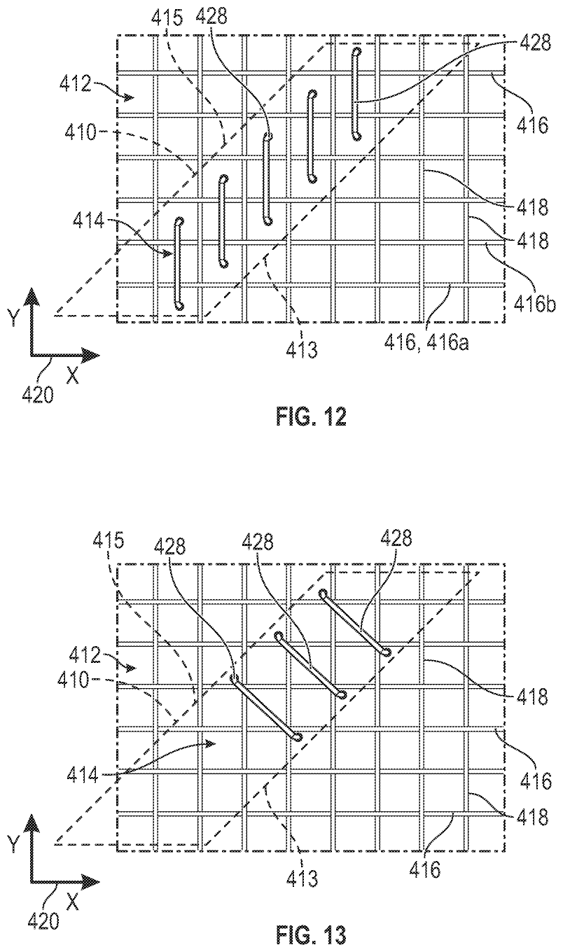

As best shown in FIG. 12, the plain weave pattern of the base layer 412 can comprise the weft yarns 418 passing over a first warp yarn 416a and then under a second warp yarn 416b in an alternating pattern. The warp yarns 416 can extend parallel to the X-axis, as shown by coordinate system 420, and the weft yarns 418 can extend parallel to the Y-axis such that the warp and weft yarns 416, 418 are perpendicular to one another.

In some embodiments, the density of the first yarns 416 can be from about 10 yarns per inch to about 200 yarns per inch, about 50 yarns per inch to about 200 yarns per inch, or about 100 yarns per inch to about 200 yarns per inch. In some embodiments, other weave patterns, for example, braided patterns, may also be used, such as “over two under two,” “over two under one,” etc. The base layer 412 may also be woven in plain weave derivative patterns such as twill, satin, or combinations of any of these.

In some embodiments, such as shown in FIG. 10, the yarns 416, 418 of the base layer 412 can be disposed at a 45-degree angle relative to an inflow edge 438 of the base layer 412. This configuration can advantageously minimize, inhibit, or prevent unravelling of the base layer 412. In some embodiments, the sutures coupling the cushioning layer 414 to the base layer 412 can likewise be angled, for example, in the same direction as either the warp or weft yarns 416, 418 of the base layer 412. Such a configuration can further facilitate the axial elongation of the sealing member 410 during compression of the prosthetic valve 400.

In some embodiments, in lieu of or in addition to a woven fabric, the base layer 412 can comprise a braided fabric, which can facilitate axial extension of the base layer 412. A braided fabric can provide various advantages including, for example, the yarns can be oriented at a non-perpendicular angle relative to one another, additional flexibility is provided with respect to the spacing of the yarns, and a braided fabric can be tubular by nature, meaning that no additional mechanism is needed to form the sealing member into a tube or cylinder. As mentioned previously, the braided fabric can comprise, for example, an “over two under two” or “over two under one” braided pattern.

As shown in FIG. 11, the cushioning layer 414 can comprise a first woven portion 422, a textured or floating yarn portion 424, and a second woven portion 426. The floating yarn portion 424 can be disposed between the first and second woven portions 422, 426 such that the first woven portion 422 can extend along the upper edge of the floating yarn portion 424, and second woven portion 426 can extend along the lower edge of the floating yarn portion 424. In this manner, the floating yarn portion 424 can be bounded or edged in a direction along the x-axis by the first and second woven portions 422 and 426. The floating yarn portion 424 can comprise a plurality of texturized yarns 428 that curve radially outwards, forming a “puffy” configuration.

The yarns 428 can extend between the first and second woven portions 422, 426. In the illustrated embodiment, the yarns 428 extend in only one direction (e.g., parallel to the longitudinal axis of the prosthetic valve 400). However, in other embodiments (see e.g., FIG. 12), the yarns 428 can extend between the first and second woven portions at an angle. For example, the yarns can be positioned at an angle of between about 10 degrees and about 20 degrees relative to the first and/or second woven portions. In some embodiments, a first portion of yarns can extend in a first direction (e.g., parallel to the longitudinal axis of the valve 400) and a second portion of yarns can extend in one or more various other directions (e.g., at an angle relative to the woven portions). In other embodiments, various yarns 428 can extend in any of various directions.

In some embodiments, the cushioning layer 414 can comprise multiple layers of yarns 428 (e.g., on top of each other). For example, in a particular embodiment, the cushioning layer 414 can comprise a first layer of yarns extending at a first angle, and a second layer of yarns disposed over the first layer of yarns and extending at a second angle.

In certain embodiments, the sealing member 410, and in particular the floating yarn portion 424, can be resiliently stretchable between a first, natural, or relaxed configuration corresponding to the radially expanded state of the prosthetic valve, and a second, elongated, or tensioned configuration corresponding to the radially compressed state of the prosthetic valve.

In some embodiments, additionally or alternatively, the yarns 428 can be sized such that they abut the base layer 412 when the prosthetic valve 400 is in a crimped configuration and such that they extend radially away from the base layer 412 when the prosthetic valve 400 is in the expanded configuration (e.g., to create a “puffy” configuration). Further details regarding various weave patterns and techniques for creating cushioning layers are disclosed in U.S. Publication 2019/0192296, which is incorporated herein by reference in its entirety. Any of the weave patterns and/or techniques described in the prior document can be used to create the cushioning layer 414.

The yarns of the base layer 412 and the cushioning layer 414 can comprise any of various biocompatible thermoplastic polymers such as PET, Nylon, ePTFE, UHMWPE, etc., or other suitable natural or synthetic filaments. In certain embodiments, one or more layers 412, 414 can be woven on a loom, and can then be heat-treated or heat-set to achieve the desired size and configuration. For example, depending upon the material selected, heat-setting can cause the layer 412, 414 to shrink. Heat-setting can also cause a texturizing effect, or increase the amount of texturizing, of the yarns (e.g., the texturized yarns 428). In some embodiments, heat setting can also induce thrombogenic characteristics to the polymer surface, which may be beneficial for PVL sealing.

As shown in FIGS. 12-13, during assembly of the sealing member 410 (the outline of which is shown in dotted lines), the cushioning layer 414 can be disposed on top of the base layer 412 for example, by placing the cushioning layer 414 on top of the base layer 412 or by sewing the yarns 428 of the cushioning layer 414 directly to the base layer 412, such that the yarns 428 are disposed in any of various orientations. For example, FIG. 12 shows an embodiment wherein the plurality of yarns 428 are oriented parallel to the weft yarns 418 (e.g., parallel to the Y-axis as shown by coordinate system 420) but are disposed at an angle relative to the inflow and/or outflow edges 413, 415 of the sealing member 410 (as shown by the outline of the sealing member 410). In the embodiment shown in FIG. 13, the yarns 428 of the cushioning layer 414 are oriented such that they extend at an angle relative to the weft and warp yarns 416, 418 but are perpendicular to the inflow and/or outflow edges 413, 415 of the sealing member 410. In particular embodiments, the yarns 428 extend at a 45-degree angle relative to the weft and warp yarns 416, 418. Such configurations advantageously allows the yarns 428 to elongate when the prosthetic valve 400 is compressed even if the yarns do not comprise an elastic material. Additionally, in FIG. 13, the yarns 416, 418 of the base layer are oriented at an angle between 0 and 90 degrees (e.g., 45 degrees) relative to the inflow and outflow ends of the frame to facilitate elongation of the base layer when the prosthetic valve is radially compressed. In other embodiments, the yarns 428 can be disposed at other angles relative to the yarns 416, 418 and the inflow and outflow ends of the frame.

Once the two layers are formed or otherwise coupled together, the sealing member 410 can be cut (e.g., from the base layer 412 by removing the excess portions of the base layer 412) such that the edges 413, 415 of the sealing member 410 are not parallel to the weft and/or warp yarns 416, 418 of the base layer 412. In other words, once the cushioning layer 414 is secured to the base layer 412 the portions of the base layer extending beyond the cushioning layer are cut to the desired shape to form the sealing member 410. This configuration allows the sealing member 410 to elongate or contract axially when the prosthetic valve is radially expanded and/or compressed. The sealing member 410 can be resiliently stretchable between a first, natural width corresponding to a non-tensioned state, and a second width when the sealing member is stretched in the Y-direction.

In some embodiments, as shown in FIGS. 12-13, the cushioning layer 414 can be woven directly onto the base layer 412 and the sealing member 410 can be cut out from the resulting fabric. In other embodiments, the cushioning layer 414 can be formed as a separate layer and subsequently coupled to the base layer 412 (e.g., using sutures), to form the sealing member 410.

Once assembled, the sealing member 410 can be mounted on the frame 402 in the following exemplary manner. Referring to FIG. 10, an outflow edge 430 of the base layer 412 can be coupled to the struts 404 of the frame 402 using one or more sutures 432. In the illustrated embodiment, the outflow edge 430 of the base layer 412 comprises a plurality of projections 434 that facilitate attachment of the base layer 412 to the frame 402. For example, the projections 434 can correspond to the zig-zag shape of the struts 404, as shown in FIG. 9. In some embodiments, the sutures 432 can extend relatively loosely around the struts 404 of the frame 402, allowing the sutures 432 to slide along the struts 404 during expansion and/or compression of the prosthetic valve 400.

In the illustrated embodiment of FIG. 9A, an inflow edge 438 of the base layer 412 can be coupled to the inflow end portion 406 of the frame 402, such as with sutures connecting the inflow edge 438 to struts of the frame along the inflow end of the frame. However, in other embodiments, as shown in FIG. 9B, an inflow edge 436 of the cushioning layer 414 can be coupled to the frame 402, for example, by suturing the inflow edge 436 of the cushioning layer to the inflow crowns or apices 437 of the frame 402 (and the inflow edge 438 of the base layer 412 need not be sutured to the frame). Such a configuration, with the outflow edge 430 of the base layer 412 and the inflow edge 436 of the cushioning layer 414 coupled to the frame 402, facilitates elongation of the sealing member during radial compression of the prosthetic heart valve 400.

In some embodiments, the cushioning layer 414 can comprise an elastic fabric configured to elongate axially, as described in more detail below. In such embodiments, the outflow edge 440 (see e.g., FIG. 11) of the cushioning layer 414 can also comprise one or more projections similar to projections 434 of the base layer 412. The outflow and inflow edges 440, 436 of the cushioning layer 414 can be sutured to the frame 402, and the base layer 412 can be attached only to the cushioning layer 414 such that it “floats” over the frame 402.

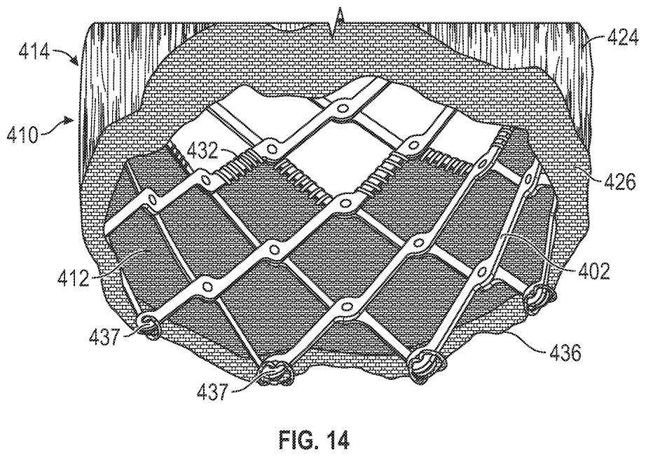

In some embodiments, such as shown in FIGS. 9B and 14, the cushioning layer 414 can be coupled to the base layer 412 such that an inflow edge 436 of the cushioning layer 414 is offset from an inflow edge 438 (FIG. 10) of the base layer 412 and the inflow end of the frame in an upstream direction (i.e., the cushioning layer 414 extends past the base layer 412 and the frame in the upstream direction). In such embodiments, the cushioning layer 414 can provide paravalvular leakage (PVL) sealing functionality when extending into the left ventricular outflow tract (LVOT).

Referring still to FIG. 14, when the sealing member 410 is mounted on the frame 402, the base layer 412 is disposed between the cushioning layer 414 and the frame 402. Such a configuration advantageously promotes radially outward thrombus formation along the cushioning layer 414, thereby preventing or mitigating PVL, while preventing or inhibiting radially inward thrombus formation toward the frame and/or valvular structure.

In certain embodiments, the loops, texturized filaments, yarns, floating yarn or filament portions, etc., of the sealing member 410 described herein can be configured to promote a biological response in order to form a seal between the prosthetic valve and the surrounding anatomy. In certain configurations, the sealing members described herein can be configured to form a seal over a selected period of time. For example, in certain embodiments, the open, porous nature of the loops, texturized filaments, yarns, etc., can allow a selected amount of paravalvular leakage around the prosthetic valve in the time period following implantation. The amount of paravalvular leakage past the sealing member may be gradually reduced over a selected period of time as the biological response to the loops, filaments, yarns, etc., causes blood clotting, tissue ingrowth, etc.

In some embodiments, the sealing member(s), and in particular the loops, filaments, yarns, etc., of the sealing member, may be treated with one or more agents that inhibit the biological response to the sealing structures. For example, in certain embodiments, the loops, filaments, yarns, etc., may be treated with heparin. In certain embodiments, the amount or concentration of the agent(s) may be selected such that the agents are depleted after a selected period of time (e.g., days, weeks, or months) after valve implantation. As the agent(s) are depleted, the biological response to the loops, filaments, yarns, etc., of the sealing structures may increase such that a paravalvular seal forms gradually over a selected period of time. This may be advantageous in patients suffering from left atrial remodeling (e.g., due to mitral regurgitation), by providing an opportunity for the remodeling to reverse as regurgitation past the prosthetic valve is gradually reduced. In some embodiments, the base layer 412 can be treated with one or more agents configured to inhibit thrombus formation.

In some embodiments, one or more layers of the sealing member 410 can comprise an elastic fabric 500. For example, the base layer 412, the cushioning layer 414, or both can be configured as an elastic fabric 500.

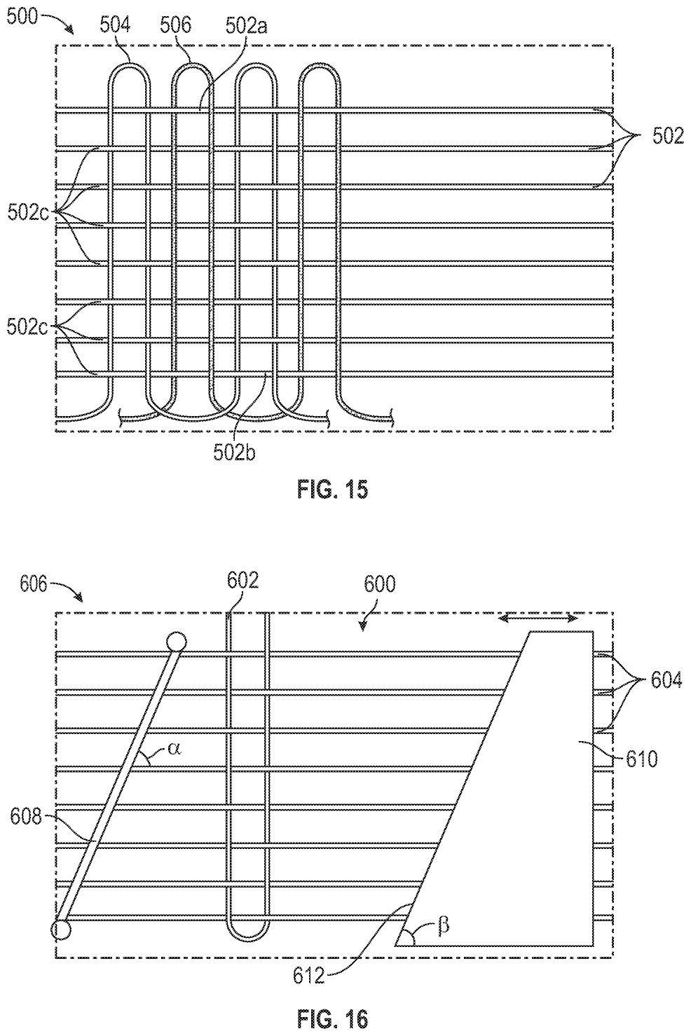

Referring now to FIG. 15, the elastic fabric 500 can comprise a plurality of warp yarns 502, a first set of weft yarns 504, and a second set of weft yarns 506. As shown in the illustrated embodiment, during weaving of the fabric 500, the first set of weft yarns 504 and the second set of weft yarns 506 can each comprise an elongated continuous yarn. Once the weaving process is complete, the fabric 500 can be cut into a selected shape, thereby cutting the first and second weft yarns into respective first and second sets of yarns 504, 506.

The first set of weft yarns 504 can be configured as elastic yarns made of, for example, implantable thermoplastic polyurethane (TPU), and/or high shrinkage yarns or highly textured yarns which can provide stretchability. The second set of weft yarns 506 can be configured as textured yarns made of, for example, implantable polyester (PET), and/or co-polymers of polyesters. The textured yarns 506 can be movable between a first or relaxed state, in which the textured yarn 506 has a wavy, curly, and/or fuzzy texture, and an elongated or stretched state in which the textured yarn 506 has a comparatively smooth texture. In some embodiments, the textured yarns can be formed via pin texturizing. In other embodiments, the textured yarns can be formed via draw texturizing, gear texturizing, and/or air texturizing.

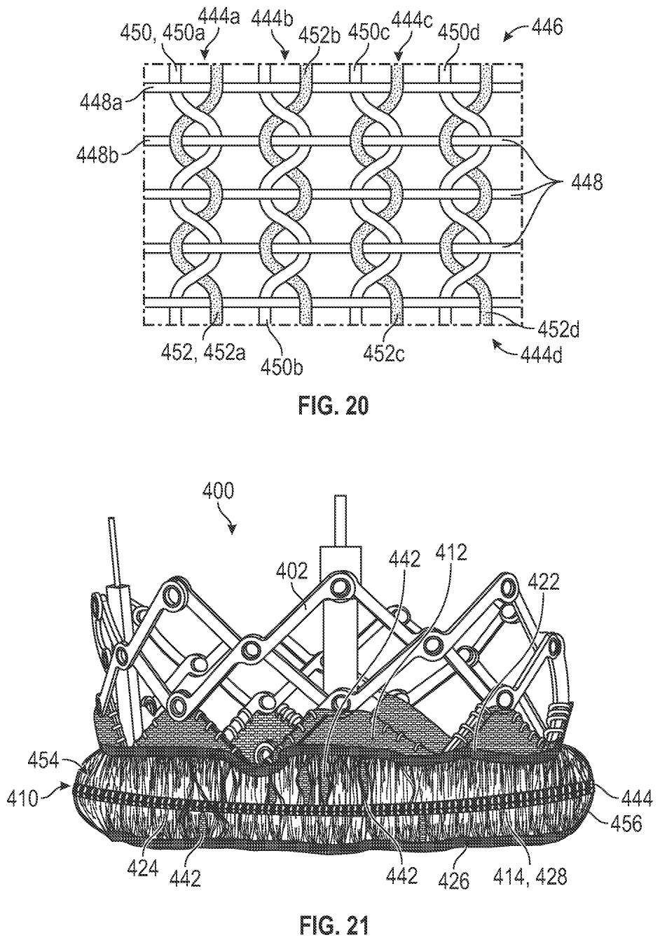

The fabric 500 can be formed by interlacing the first set of weft yarns 504 with the warp yarns 502 such that they extend over and under the warp yarns 502 in an alternating woven pattern. The second set of weft yarns 506 can be configured as “floating” yarns, meaning that they extend over the warp yarns 502 without interlacing. The floating yarns 506 can be anchored at either end by extending around one or more warp yarns 502. For example, in the illustrated embodiment, the floating yarns 506 extend around a first end warp yarn 502a and a second end warp yarn 502b, but extend over intermediate warp yarns 502c without interlacing. In some embodiments, the first and second end yarns 502a, 502b can be configured as leno weaves (see e.g., FIG. 20) to strengthen the edges of the fabric 500. In the illustrated embodiment, all floating yarns 506 extend over the same surface of the warp yarns 502. That is, in the illustrated embodiment, all the floating yarns 506 are on top of the warp yarns 502 in the orientation shown in FIG. 15. However, in other embodiments, some or all of the floating yarns 506 can be beneath the warp yarns 502. As mentioned previously, in some embodiments, each yarn can comprise a plurality of filaments and in other embodiments each yarn can be a monofilament yarn.

When a sealing member 410 comprising a fabric 500 is coupled to the frame 402 of a prosthetic valve 400, the fabric 500 can be disposed such that the floating yarns 506 are oriented radially outwardly of the warp yarns 502. Such a configuration advantageously promotes radially outward thrombus formation along the radially outer surface of the fabric 500, thereby preventing or mitigating PVL while preventing or inhibiting radially inward thrombus formation. In some embodiments, the non-textured yarns (e.g., warp yarns 502 and elastic yarns 504) can be coated with an anti-thrombogenic agent or otherwise provided with an anti-thrombogenic surface.

The fabric 500 can be coupled to the frame 402 via the warp yarns 502 and/or the elastic yarns 504. The elastic yarns 504 allow the fabric 500 to elongate or contract axially (e.g., longitudinally) when the prosthetic valve 400 moves between the expanded configuration and the compressed or crimped configuration.

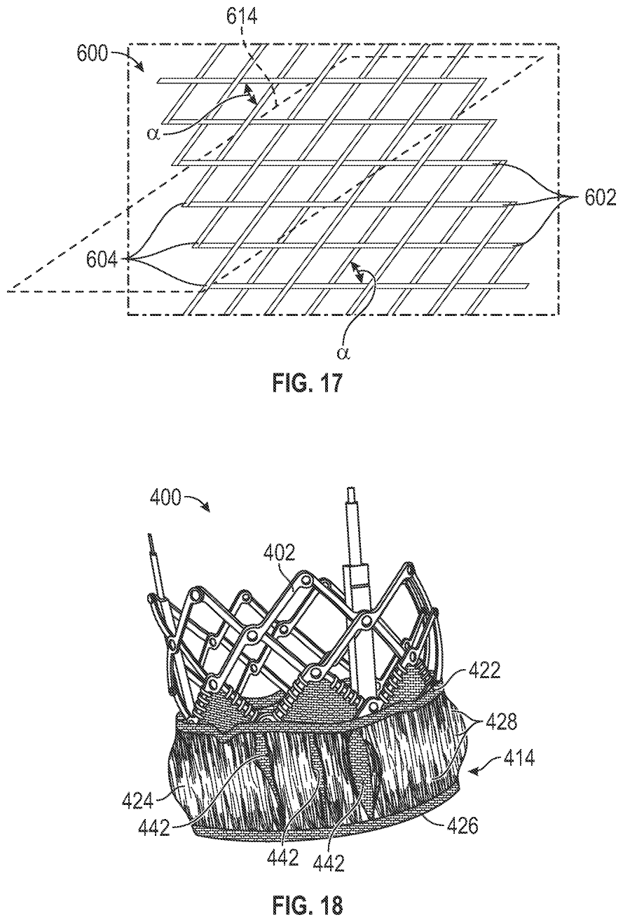

Referring now to FIGS. 16-17, in some embodiments, one or more layers of the sealing member 410 can comprise an angled weave fabric 600 (FIG. 17) comprising first and second sets of yarns disposed at a non-perpendicular angle relative to one another. For example, the base layer 412, the cushioning layer 414, or both can be configured as an angled weave fabric 600.

As shown in FIG. 17, the angled weave fabric 600 can have a first set of yarns 602 and a second set of yarns 604. The first and second sets of yarns 602, 604 can be disposed at a non-perpendicular angle α relative to one another. For example, in the illustrated embodiments, the angle α between the first set of yarns 602 and the second set of yarns 604 can be 45 degrees. The angle α between the second set of yarns 604 and the first and second edges of the fabric 600 can be 45 degrees.

An exemplary method of weaving an angled weave fabric 600 can proceed as follows. FIG. 16 illustrates a loom 606 comprising a plurality of warp yarns or yarns 604. The weft yarns or yarns 602 can be interlaced with the warp yarns 604 at a conventional perpendicular angle relative to the warp yarns 604, as shown in FIG. 16. The loom 606 can include a base member 608 which is coupled to the loom 606 at an angle α relative to the warp yarns 604. The loom 606 can further comprise a loom reed or pusher bar 610 configured to push the weft yarns 602 against the base member 608, thereby angling the weft yarns 602 relative to the warp yarns 604. The movable loom reed 610 can comprise an angled pushing surface 612 having an angle β that corresponds to the angle α of the base member 608. This configuration advantageously allows the weft yarns 602 to be woven through the warp yarns 604 perpendicularly, which is simpler to achieve, while still resulting in an angled weave fabric 600 wherein the weft and warp yarns 602, 604 are positioned at a non-perpendicular angle to one another.

In another embodiment, the fabric can be formed by weaving the weft yarns 602 through the warp yarns 604 at a desired non-perpendicular angle α on the loom 606.

As shown in FIG. 17, one or more layers of the sealing member 410 can be cut from the angled weave fabric 600, as shown by the dotted line 614. The edges of the sealing member 410 can be cut at a predetermined angle relative to the yarns 602, 604. In other words, the cut portion 614 can be oriented at a desired angle relative to the angled weave fabric 600 to produce selected characteristics of the sealing member 410, such as selected elongation characteristics. For example, an angled weave fabric 600 formed from non-perpendicular weft and warp yarns 602, 604 can undergo increased axial elongation during compression of a prosthetic valve, when compared to a fabric having perpendicular warp and weft yarns.

As shown in FIG. 18, in some instances, the sealing member 410 can comprise one or more gaps 442 in the cushioning layer 414. The gaps 442 can interfere with the PVL sealing function of the sealing member 410 by, for example, forming channels for blood to flow through the cushioning layer 414 and around the prosthetic valve 400. The gaps 442 can form where the plurality of filaments 428 of the cushioning layer 414 bunch toward and/or extend away from one another circumferentially. The gaps 442 can, for example, extend from the first woven portion 422 to the second woven portion 426 of the cushioning layer 414.

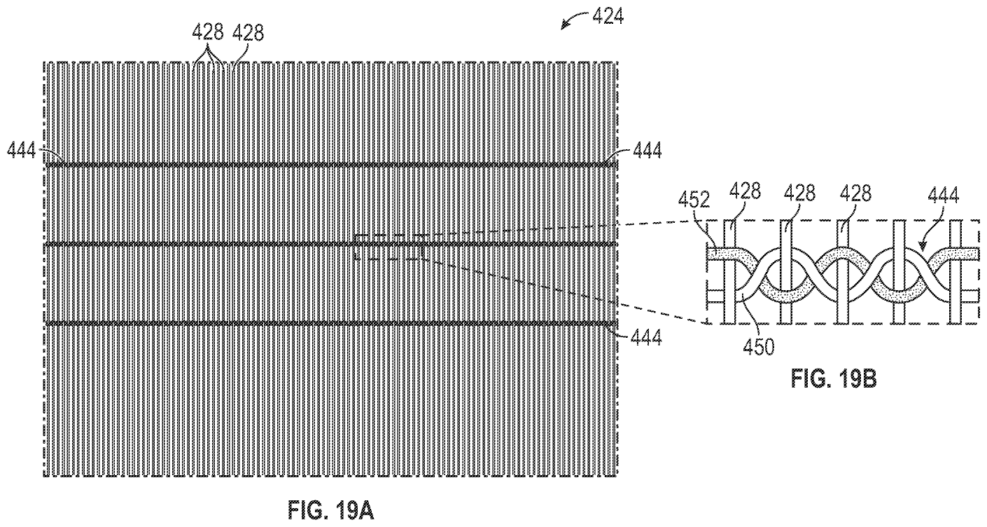

Referring now to FIGS. 19A-21, in some embodiments, the floating yarn portion 424 of the cushioning layer 414 can comprise one or more leno weave portions 444 (see e.g., FIG. 19B). Each leno weave portion 444 can extend circumferentially around at least a portion of the sealing member 410 and can be configured to prevent or mitigate the propagation of gaps within the cushioning layer 414. FIG. 20 illustrates an exemplary leno weave 446 including four leno weave portions 444a-444d. A leno weave comprises a set of warp yarns 450, 452 that interlaces with weft yarns 448. In FIGS. 19A and 19B, the weft yarns 448 are the filaments 428 of the cushioning layer 414. Referring to weave portion 444a in FIG. 20 as an exemplary portion, a first leno yarn 450a can pass under a first weft yarn 448a, cross over the second leno yarn 452a, and pass under a second weft yarn 448b. The adjacent leno yarn 452a can pass over the first weft yarn 448a, cross under the first leno yarn 450a, and pass under the second weft yarn 448b. This pattern can continue indefinitely until a selected length of the leno portion 444a is reached. Each weft yarn 448 is trapped between respective first and second leno yarns 450, 452, thereby mitigating circumferential motion of the weft yarns 448. Details of various other leno weaves are disclosed in U.S. Publication 2019/0192296.

In some embodiments, each weft yarn 448 can comprise multiple filaments bunched together. The number of filaments that make up a yarn can be between 1 and about 200.

The leno portion 444 can extend circumferentially around at least a portion of the frame 402 using a plurality of yarns 428 as the weft yarns 448. The leno warp yarns 450, 452 (FIG. 19B) hold or trap the plurality of yarns 428 in a selected position relative to one another, such that the plurality of yarns 428 cannot move toward or away from each other at the locations where they are trapped by the leno yarns 450, 452, thereby mitigating the formation and/or size of the gaps 442.

As shown in FIG. 21, the leno weave portion 444 can divide the floating yarn portion 424 into a first or upper portion 454 extending between the leno weave portion 444 and the first woven portion 422, and a second or lower portion 456 extending between the leno weave portion 444 and the second woven portion 426. The leno weave portion 444 is configured to stop the propagation of one or more gaps 442 such that the gaps 442 are contained within the upper and/or lower portions 454, 456 and cannot extend over or past the leno weave portion 444 as a continuous gap. In this way, the leno weave portion 444 advantageously limits the size of any gaps 442 that form.

In some embodiments, such as shown in FIG. 19A, the floating yarn portion 424 can comprise a plurality of leno weave portions 444 (e.g., three) configured to further reduce the size of any gaps 442 that may form. In such embodiments, gaps 442 may form between the first and second woven portions 422,426 (see FIG. 21) and a respective leno weave portion 444, and or between two adjacent leno weave portions 444.

In the illustrated embodiment, the leno weave portion 444 extends circumferentially around the frame 402 in a continuous line substantially perpendicular to a longitudinal axis of the prosthetic valve 400. However, in other embodiments, the leno weave portion 444 can have any of various shapes. For example, in some embodiments, the leno weave portion 444 can extend circumferentially around the frame 402 in a continuous zig-zag or sinusoidal shape. In other embodiments, the leno weave portion 444 can extend around the circumference of the frame 402 as a series of discontinuous portions, for example, a series of discontinuous overlapping portions such as a plurality of overlapping steps. In still other embodiments, the leno weave portion 444 need not extend entirely around the circumference of the frame 402 and extends around only a portion of the circumference.

In some embodiments, the leno yarns 450, 452 can be configured as extendible, elastic, and/or flexible yarns made of, for example, implantable thermoplastic polyurethane (TPU), and/or high shrinkage yarns or highly textured yarns which can provide stretchability. In such embodiments, the leno weave portion 444 can expand with the prosthetic valve 400 as the prosthetic valve moves from the compressed configuration to the expanded configuration. As mentioned previously, the weft yarns 448 can be configured as textured yarns made of, for example, implantable polyester (PET), and/or co-polymers of polyesters.

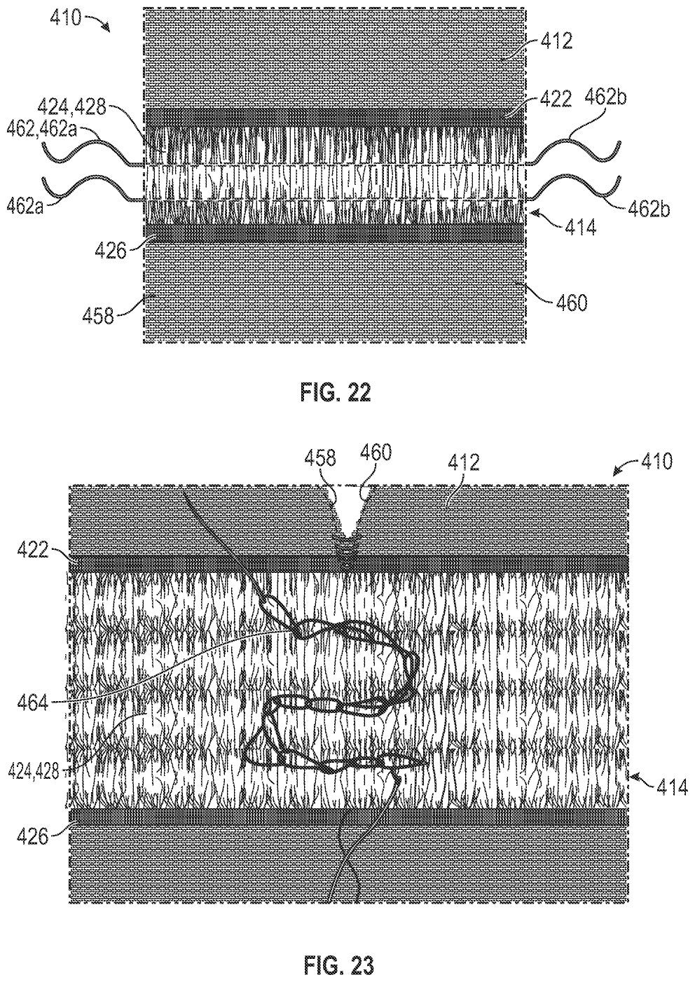

In some embodiments, prior to being coupled to the frame 402, the sealing member 410 can be configured as a cylinder or tube by coupling first and second circumferential edges 458, 460 (see e.g., FIG. 22) of the sealing member 410 to one another.

As shown in FIG. 22, one or more elongated yarns 462 can be woven or stitched through the cushioning layer 414 (e.g., using a running stitch, whip stitch, back stitch, zig-zag stitch, etc.). Each elongated yarn 462 can have a first extending portion 462a and a second extending portion 462b which extend past the respective circumferential edges 458, 460 of the sealing member 410. Each first extending portion 462a can be tied to a respective second extending portion 462b such that the sealing member 410 forms a tube. This configuration advantageously mitigates bunching between the first and second circumferential edges 458, 460, thereby mitigating the risk of blood leakage through such regions. While the illustrated embodiment shows two elongated yarns 462, any number of yarns 462 can be used. For example, in some embodiments, the sealing member 410 can comprise one, three, four, five, or six elongated yarns 462. In some embodiments, the elongated yarns 462 can be configured as leno weaves, such as described previously with respect to FIGS. 16-19. In such embodiments, the elongated yarns 462 can further function to prevent or mitigate gap formation, as described previously.

In the illustrated embodiment, the elongated yarns 462 extend through the floating yarn portion 424 of the cushioning layer 414. However, in other embodiments, the elongated yarns 462 can extend through any of various components of the sealing member 410. For example, the elongated yarns 462 can be woven or stitched through the base layer 412, first or second woven layers 422, 426, and/or can extend between the base layer 412 and the cushioning layer 414.

In some embodiments, the elongated yarns 462 can be configured as elastically stretchable yarns made of, for example, implantable thermoplastic polyurethane (TPU), and/or high shrinkage yarns or highly textured yarns which can provide stretchability. In such embodiments, the elongated yarns 462 can expand with the prosthetic valve 400 as the prosthetic valve moves from the compressed configuration to the expanded configuration.

Referring now to FIG. 23, in other embodiments, the first and second axially extending edges 458, 460 can be coupled together using a chain stitch 464. In some embodiments, the chain stitch 464 can be configured to extend only partially through the thickness of the sealing member 410, such that the chain stitch 464 is only visible from one side of the sealing member 410. Such a configuration can advantageously mitigate the risk of diminishing the sealing characteristics of the sealing member 410.

For example, the chain stitch 464 can be sewn on the inner surface of the sealing member 410. In another example, the chain stitch 464 is sewn on the outer surface of the sealing member 410. The chain stitch 464 can extend through one or more layers of the sealing member 410. For example, in some embodiments, the chain stitch 464 extends only through the base layer 412. In other embodiments, the chain stitch 464 extends only through the cushioning layer 414. In still other embodiments, the chain stitch 464 can extend entirely through the base layer 412 and through only a portion of the cushioning layer 414, or vice versa.

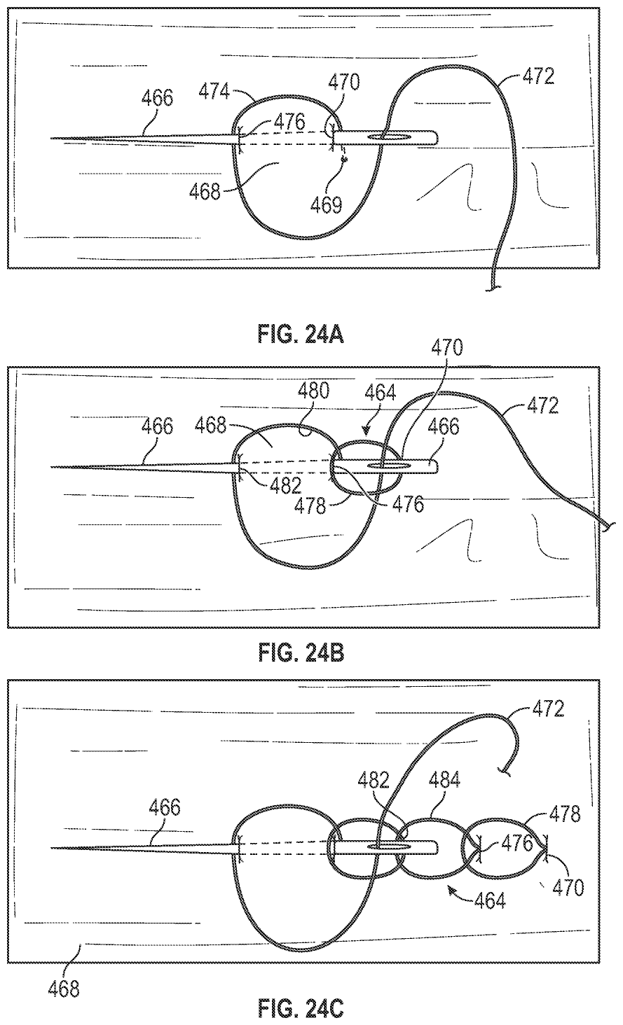

FIGS. 24A-24C illustrate an exemplary chain stitching technique that can be used to form the chain stitch 464, which can be used to form the sealing member 410, or any other sealing member described herein, into a cylinder. As shown in FIGS. 24A-24C, a needle 466 coupled to a suture, thread, or yarn 472 is brought up through a material layer 468 of the sealing member from a first side (the lower side in the drawings) such that the needle 466 and the yarn 472 are on the second side (the upper side in the drawings) of the material layer 468. The end of the yarn 472 can be knotted at 469 (on the first side of the layer) to prevent it from pulling through the material layer. On the second side of the material layer 468, the assembler forms a loop 474 with the yarn 472. The assembler then pierces the material layer 468 with the needle at a first location 470 at or near the knotted end 469 on the second side of the material layer and then again at a second location 476, spaced from the first location, on the first side of the material layer, and then pulls the needle 466 and the yarn 472 through locations 470, 476 and the loop 474. Continued pulling of the needle tightens the loop 474 and forms the first link 478 (FIG. 24B) in the chain stitch 464. A second loop 480 is then formed on the second side of the material layer and the needle 466 is pushed back through the second location 476 a second time from the second side of the material layer to the first side, and then through the material layer at a third location 482, spaced from the second location 476, from the first side to the second side. The needle and the yarn is pulled through the material layer at locations 476 and 482 and the loop 480 is tightened to form the second link 484 (FIG. 24C) in the chain stitch 464. This process can repeated as necessary to form additional links until the chain stitch 464 reaches a selected length.

As shown in FIG. 23, the chain stitch 464 can be sewn in a zig-zag or sinusoidal pattern. The sinusoidal pattern can advantageously increase the area of the stitch, thereby improving the strength and stability of the chain stitch 464. The chain stitch 464 can pass over the joint where the first and second edges 458, 460 abut multiple times to strengthen the coupling between the two edges 458, 460. In the illustrated embodiment, the chain stitch 464 passes over the joint three times, however, in other embodiments, the chain stitch 464 can pass over the joint any number of times.

In some embodiments, the sealing member 410 can be formed into a cylindrical or tubular shape, such as by using the chain stitching technique or other techniques described above, before placing the sealing member on a frame of a prosthetic valve. After forming the tubular shaped sealing member, the sealing member can be slid onto the outer surface of the frame and secured to the frame, such as with sutures. In other embodiments, prior to attaching the opposing edges of the sealing member 410, the sealing member can be wrapped around the outer surface of the frame and the opposing edges can be attached to each other, using the chain stitching technique or other techniques described above, thereby forming the tubular shape of the sealing member around the frame. The sealing member can then be secured to the frame, such as with sutures.

The above-described sealing members and/or skirts can be coupled to the frame of a prosthetic valve using any of various methods. Typically, when attaching a sealing member to a prosthetic valve, it is desirable that the yarns of the sealing member be aligned with the struts of the prosthetic valve. As used herein, the term “aligned with” means parallel to an axis extending longitudinally through a component. For example, a yarn is aligned with a strut if the yarn is oriented parallel to an axis extending longitudinally through the strut (e.g., the “geometric centerline” of the strut).

For balloon expandable prosthetic valves, it can be desirable to couple the sealing member (e.g., a sealing member having warp and weft yarns oriented perpendicularly to one another) to the prosthetic valve such that the two sets of yarns extend at 45 degree angles relative to the inflow and/or outflow edges of the prosthetic valve in the expanded state. For mechanically expandable valves (e.g., prosthetic valves 10 and 400 described above), it can be advantageous to attach the sealing member to the frame when the struts of the frame are perpendicular to one another. In some embodiments, for example, the struts can be perpendicular to one another when the prosthetic valve is in a partially expanded configuration.

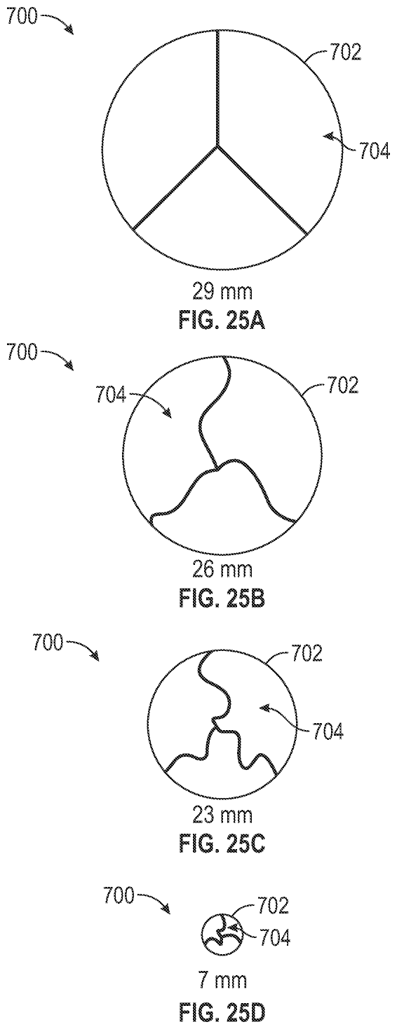

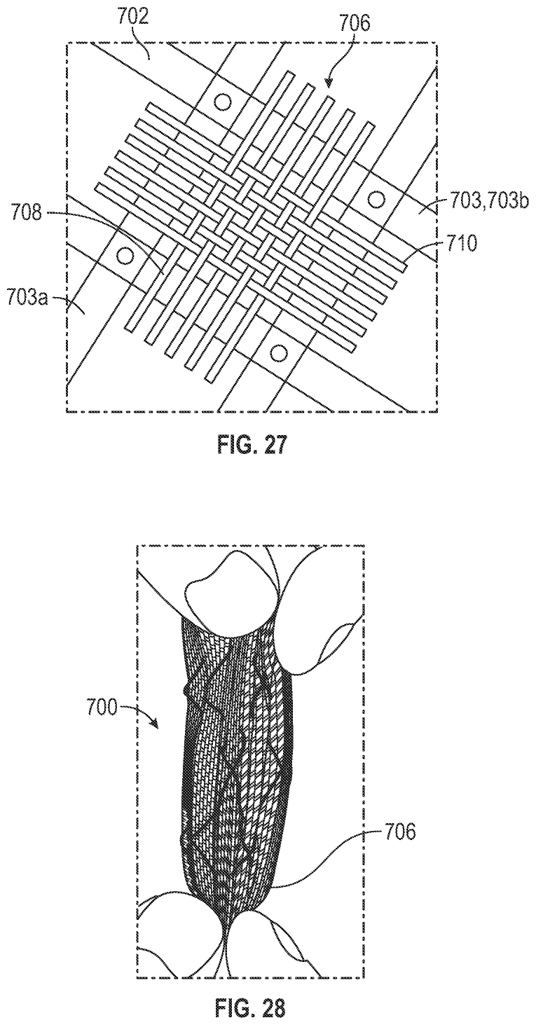

FIGS. 25A-25D illustrate a particular embodiment of a mechanically expandable prosthetic valve 700 in various states expansion and compression. Prosthetic valve 700 can have a frame 702 comprising a plurality of struts 703 (see e.g., FIG. 27) and a valvular structure 704 comprising a plurality of leaflets. The illustrated prosthetic valve 700 can have a fully expanded diameter of 29 mm (FIG. 25A), a fully compressed diameter of 7 mm (FIG. 25D), and a working range of diameters between 26 mm and 29 mm. The working range of diameters is defined as the range of diameters at which the prosthetic valve can function when implanted within the subject, such as within a body of a patient.

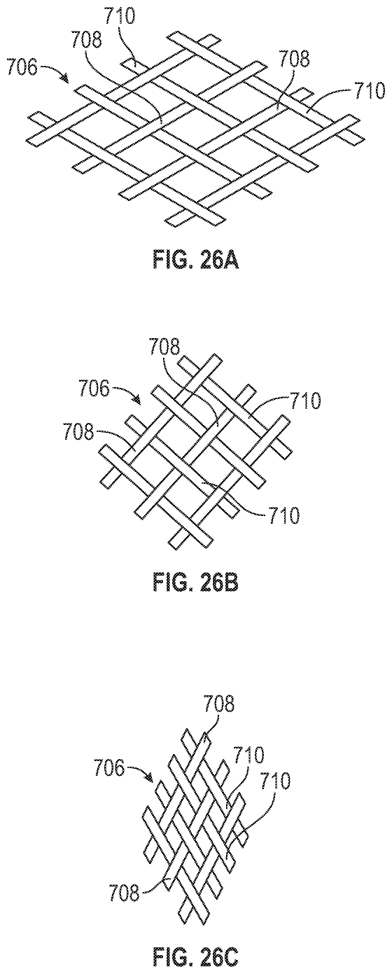

When the prosthetic valve 700 is in the fully expanded configuration, such as shown in FIG. 25A, the struts 703 of the frame 702 are at a non-perpendicular angle to one another. When the prosthetic valve 700 is in the fully compressed configuration, such as shown in FIG. 25D, the struts 703 are likewise at a non-perpendicular angle to one another. When the prosthetic valve 700 is partially expanded to a non-working diameter, such as shown in FIG. 25C, the struts 703 are positioned perpendicularly to one another. Accordingly, when the sealing member 706 (see e.g., FIGS. 26A-26C) is disposed over the partially expanded frame, as shown in FIG. 27, the warp and weft yarns 708, 710 of the sealing member 706 are respectively aligned with (e.g., parallel to an axis extending longitudinally through) the first and second sets of struts 703a, 703b.

Once attached to the prosthetic valve 700, the sealing member 706 can expand as the prosthetic valve moves between the expanded and compressed configurations. Referring now to FIGS. 26A-26C, as the prosthetic valve 700 is expanded from the partially expanded configuration (FIG. 26B) to the fully expanded configuration, the sealing member 706 can stretch or elongate circumferentially, as shown in FIG. 26A, such that the warp and weft yarns 708, 710 are no longer perpendicular to one another. As the prosthetic valve 700 is compressed from the partially expanded configuration (FIG. 26B) to the fully compressed configuration, the sealing member 706 can elongate in the axial direction, as shown in FIG. 26C, such that the warp and weft yarns 708, 710 are no longer perpendicular to one another.

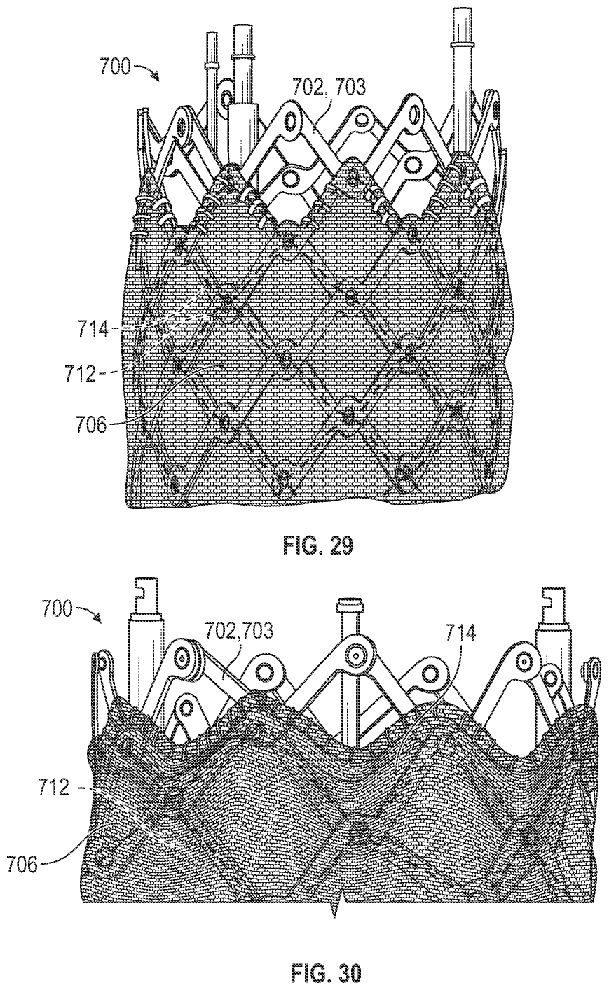

FIG. 28 illustrates the prosthetic valve 700 in the fully compressed configuration (e.g., having a diameter of 7 mm), with the yarns 708, 710 of the sealing member 706 elongated axially (see FIG. 26C). FIG. 29 illustrates the prosthetic valve 700 in the partially expanded configuration (e.g., having a diameter of 23 mm), with the sealing member 706 in an unstretched position, such that the yarns 708, 710 (the orientations of which are indicated by dotted lines 712, 714, respectively) of the sealing member 706 align with the struts 703 of the prosthetic valve 700. FIG. 30 illustrates the prosthetic valve 700 in a fully expanded configuration (e.g., 29 mm), such that the yarns 708, 710 of the sealing member are circumferentially elongated, as shown by the corresponding dotted lines 712, 714.

In some embodiments, as shown in FIGS. 28-30, the sealing member 706 can comprise one or more indicators, such as markings represented by dashed lines 712, 714, indicating the direction of the sealing member yarns 708, 710. Such indicia can help an assembler align the sealing member 706 with the frame 702 during assembly of the prosthetic valve 700. The markings can be in the form of dashed lines as shown, solid lines, or can have various other shapes or forms. The markings can be, for example, ink markings made on the sealing member or yarns having different colors. For example, the yarns 708 or selected ones of yarns 708 can have a first color and the yarns 710 or selected ones of yarns 710 can have a second color. As another example, additional threads or yarns optionally having a different color than yarns 708, 710 can be stitched in the skirt at selected locations to indicate the orientation of the yarns 708, 710.

In some embodiments, in lieu of or in addition to woven fabric, such as shown in sealing member 706 of FIGS. 26A-30, the sealing member can comprise a braided fabric. In such embodiments, the warp and weft yarns need not necessarily be perpendicular relative to one another. The braided fabric can be configured to have any initial angle (e.g., ‘relaxed’ or ‘free state’ angle) between its yarns.

The braided fabric can advantageously be attached to a frame when the struts of the frame are at a non-perpendicular angle to one another. For example, the braided fabric can be coupled to the frame when the frame is expanded to a half-way state (e.g., half-way between the fully compressed and fully expanded diameters). Referring to valve 700 of FIGS. 25A-25D, a half-way state of the frame would have a diameter of 11 mm. The braided fabric can be coupled to the frame such that the warp and weft yarns of the braided fabric align with the struts of the frame (e.g., are substantially parallel with a geometric centerline of a respective strut).

Once attached to the prosthetic valve, the sealing member comprising the braided fabric can expand as the prosthetic valve moves between the expanded and compressed configurations. Coupling the sealing member to the frame at a half-way state advantageously allows the inflow and outflow edges of the sealing member to move an equal distance relative to one another when the prosthetic valve is expanded and/or compressed. For example, as the prosthetic valve is expanded from the half-way state to the fully expanded state, the inflow and outflow edges of the sealing member move toward each other a first distance, and as the prosthetic valve is compressed from the half-way state to the fully expanded state the inflow and outflow edges of the sealing member move away from one another a second distance, the second distance being equal to the first distance.

In some embodiments, the braided fabric can be configured to facilitate alignment between the warp and weft yarns of the braided fabric and the struts of the frame when the frame is in the fully expanded position. In such embodiments, the sealing member can be coupled to the frame when the frame is in the fully expanded position, which advantageously facilitates the assembly process.

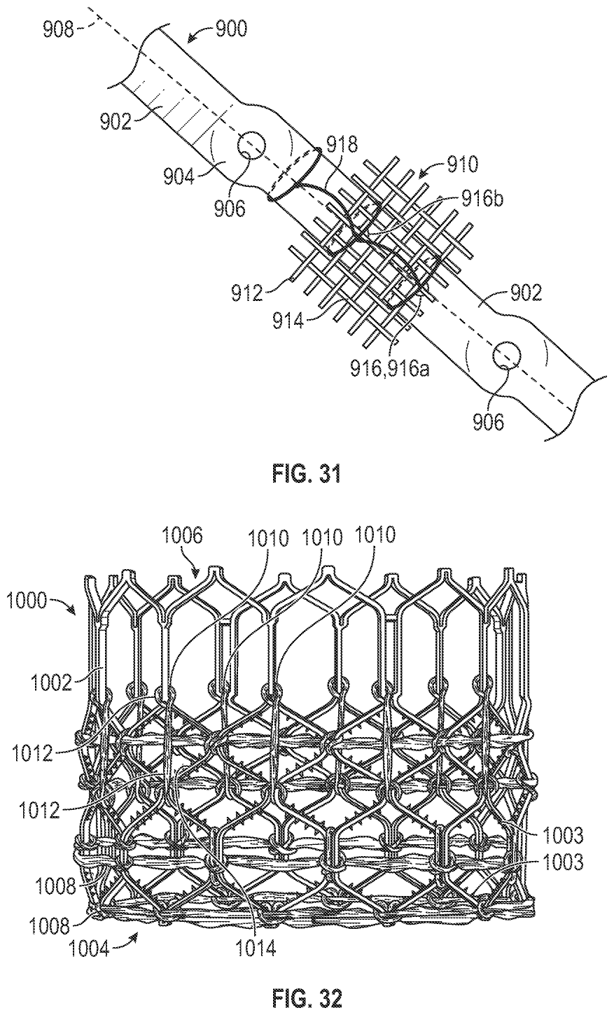

Referring now to FIG. 31, in some embodiments, the frame of a prosthetic valve (e.g., frame 12 as shown in FIG. 2B) can comprise a plurality of struts 900, and each strut can comprise a plurality of segments 902. The segments 902 can be arranged end-to-end relative to each other with adjacent end interconnected to each other by enlarged (relative to segments 902) portions 904. Each of the enlarged portions 904 can have a respective aperture 906, such as at its geometric center, for receiving a fastener to pivotably coupled one or more struts 900 together. Each segment 902 can be slightly laterally offset from one or more adjacent segments 902 in a direction perpendicular to the overall length of the strut 900. Such a strut 900 can be referred to as a “zig-zag” strut.

Each zig-zag strut 900 can comprise a geometric centerline (indicated by dashed line 908) that passes through each aperture 906. Due to the zig-zag configuration of the strut 900, the geometric centerline 908 is not necessarily positioned directly in the center of each segment 902.

In some embodiments, a skirt or sealing member 910 having a plain weave (e.g., the warp yarns 912 and weft yarns 914 are perpendicular to one another) can be coupled to a frame comprising a plurality of zig-zag struts 900 in the following exemplary manner. The sealing member 910 can be disposed over the struts 900 such that the warp yarns 912 and/or weft yarns 914 are aligned with (e.g., parallel to an axis extending longitudinally through) the geometric centerline 908 of a respective strut 900. For example, in FIG. 31, the weft yarns 914 are aligned with the geometric centerline 908. In other embodiments, the sealing member 910 can be oriented such that the warp yarns 912 are aligned with the geometric centerline 908.

The stitching used to connect a skirt to a frame can be aligned with the geometric centerlines of the struts of the frame. For example, in some embodiments, the sealing member 910 can comprise a series of apertures 916 (e.g., preformed holes in the fabric and/or gaps between filaments of the fabric) via which the sealing member 910 is coupled to the strut 900. Attachment means, such as a suture 918, can extend through a first aperture 916a, around the strut 900 and back through the first aperture 916a. The suture 918 can then extend over (e.g., radially outward of) the sealing member 910 to the second aperture 916b. For each aperture along the strut, the suture 918 can extend through the aperture, around the strut, back through the aperture and then can extend to the next adjacent aperture. The portions of the suture 918 that extend between the apertures 916 can be aligned with the geometric centerline 908 of the strut 900.

In some embodiments, the sealing member 910 can comprise one or more indicators, such as lines or other markings, indicating the direction of the yarns 912, 914 (see e.g., dashed lines 712 and 714 in FIG. 29 or other types of markings described above in connection with FIG. 29). Such indicia can help an assembler properly align the sealing member 910 with the frame during assembly of the prosthetic valve such that the yarns 912, 914 align with the geometric centerlines 908 of respective struts 900. Aligning the yarns and/or attachment means with the geometric centerlines 908 of the struts 900 prevents or mitigates the formation of “geometric triangles” between the attachment means (e.g., sutures 918) and the sealing member 910. Geometric triangles occur when the attachment means does not cross both the warp and weft yarns 912, 914, but rather runs parallel to one of the sets of yarns. The disclosed attachment configurations advantageously promote alignment between the sealing member's yarns 912, 914 and the zig-zag struts 900 of a mechanically expandable prosthetic valve.

FIG. 32 illustrates an embodiment of a prosthetic valve 1000. The prosthetic valve 1000 can comprise a frame 1002, inflow end portion 1004, and outflow end portion 1006. The prosthetic heart valve 1000 can include a valvular structure (e.g., valvular structure 18 or a valvular structure 1108, described below), and an inner skirt (e.g., an inner skirt 1110, described below), though these components are omitted for purposes of illustration. The frame 1002 can be a plastically expandable frame formed from, for example, stainless steel or a cobalt-chromium alloy, and can be radially expanded using a balloon or other expansion mechanism. Thus, the prosthetic valve 1000 can be referred to a balloon-expandable valve. Further details regarding the prosthetic valve are disclosed in U.S. Pat. No. 9,393,110, which is incorporated herein by reference. In other embodiments, the prosthetic valve 1000 can be a self-expandable valve having a frame made of a shape-memory material, such as Nitinol.

As shown in FIG. 32, the frame 1002 can comprise a plurality of tissue engagement elements, projections, or anchoring members 1003 that extend from selected struts of the frame 1002. The anchoring members 1003 can be configured to secure the prosthetic valve 1000 to native tissue at a selected implantation site and/or help promote tissue ingrowth between the native tissue and the prosthetic valve 1000. The anchoring members 1003 can extend in various directions from the struts of the frame 1002. For example, in some embodiments, some or all of the anchoring members 1003 can extend from the struts at an angle relative to a central longitudinal axis extending from the inflow end to the outflow end of the prosthetic heart valve assembly. In some instances, the anchoring members 1003 can be perpendicular or at least substantially perpendicular (e.g., forming an angle of 80-100 degrees) to the struts from which they extend. In other embodiments, the anchoring members 1003 can extend from their respective struts at various other angles (e.g., between 1-79 degrees). For example, in some embodiments, the anchoring members can extend from their respective struts at an angle of about 45 degrees such that anchoring members are parallel or at least substantially parallel to a central longitudinal axis extending from the inflow end to the outflow end of the prosthetic heart valve assembly.

The anchoring members 1003 can comprise various shapes and lengths such that the projections provide sufficient retention force for the prosthetic heart valve assembly, while reducing potential harm to the surrounding tissue. For example, in the illustrated embodiment, the anchoring members 1003 comprise tines or spikes. In other embodiments, the anchoring members 1003 can comprise ball-shaped bulges and/or a rectangular shape. Additionally or alternatively, one or more of the anchoring members can comprise a curved shape, a hook shape, a cross shape, a T-shape, and/or a barbed shape. Various combinations of shapes and/or sizes of anchoring members 1003 can be used.

Further configurations and details of the anchoring members 1003 can be found, at least, in U.S. Provisional Application No. 63/030,811 filed May 27, 2020 and entitled “Devices and Methods for Securing Prosthetic Valves” which is incorporated by reference herein in its entirety. Any of the prosthetic valves described therein can be used with the expandable sutures and/or sealing members disclosed herein.

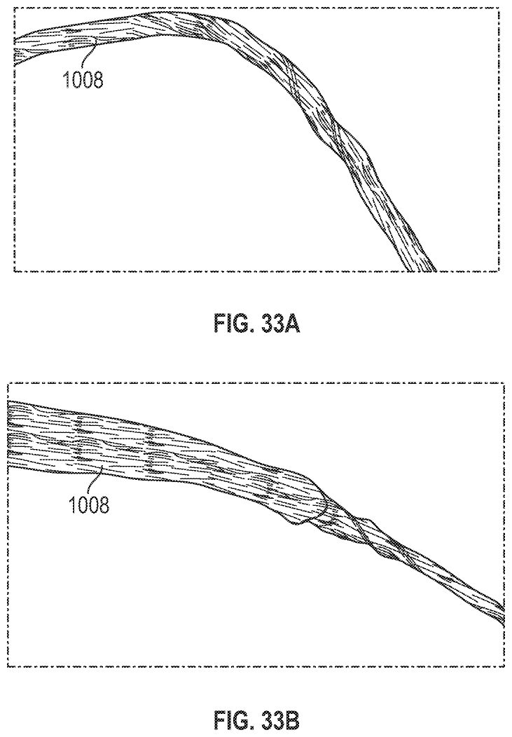

In lieu of or in addition to a sealing member or outer skirt disposed on the outside of the frame, the prosthetic valve 1000 can comprise one or more expandable yarns or sutures 1008. Referring to FIGS. 33A and 33B, a suture 1008 can be resiliently stretchable and can be placed in a tensioned and axially elongated state (FIG. 33A) when tensioned in applied and a relaxed, non-tensioned state (FIG. 33B) when tension is removed. When placed in tension, the suture 1008 is axially elongated and has a reduced diameter (FIG. 33A), but when tension is released (e.g., when the suture is in a relaxed state) the sutures can increase in diameter and become fuzzy and puffy (FIG. 33B), increasing their ability to absorb fluid (e.g., blood). The sutures 1008 can function as a sealing member for the prosthetic valve by sealing against the tissue of the native valve annulus and helping to reduce paravalvular leakage past the prosthetic valve. In some embodiments, the sutures 1008 can comprise a plurality of texturized filaments, which can be, for example, twisted or braided together. The texturized filaments can be texturized via, for example, pin texturizing. For example, in some embodiments, the expandable sutures can comprise draw texturized yarn (DTY) including a plurality of filaments that have been twisted together (e.g., 3,000-4,000 times per meter) and heat treated to create fine crimps in the filaments. In other embodiments, the filaments can be texturized via gear texturizing and/or air texturizing.

As shown in FIG. 32, the prosthetic valve 1000 comprises a plurality of sutures 1008 circumscribing the outer surface of frame 1002 and spaced apart from one another along a longitudinal axis of the prosthetic valve 1000. The prosthetic valve 1000 can further comprise vertically extending sutures 1010. The vertically extending sutures 1010 can extend between opposing junctions 1012 of a respective cell 1014 of the frame 1002. In the illustrated embodiment, a sealing member on the outer surface of the frame is omitted. The sutures 1008 cover a reduced surface area of the outer surface of the frame 1002 relative to a typical outer skirt, which advantageously allows the anchoring members 1003 to engage the native tissue.

As mentioned, in some embodiments, the sutures 1008 can comprise a plurality of texturized filaments that are combined (e.g., twisted and/or braided) together to form the expandable suture. The sutures 1008 can comprise any number of filaments, for example, between 2 and 20 filaments. In some particular examples, the sutures comprise 12 filaments. When tensioned, the sutures 1008 can have a diameter equivalent to that of a suture typically used for securing soft components of a prosthetic valve to each other or to a frame of the valve (e.g., a 2-0, 3-0, or 4-0 suture), for example, between about 0.15 mm to about 0.3 mm. In some particular embodiments, the expandable sutures 1008 can comprise polyester.

In some particular embodiments, the sutures 1008 can each comprise 8 filaments or “ends” of pin textured polyethylene terephthalate (PET). The linear mass density of the filaments can be 1/40 Den/27 Fil. The filaments can be braided together at a braid density of 10 picks per inch (PPI) with alternating carrier tension (e.g., during braiding some of the filaments can be held in tension while others are held loosely, or some filaments can be held at a first tension while others are held at a second tension, etc.) to form the suture 1008. The suture(s) 1008 can be heat set at, for example, 320° F. while wrapped around a spool. In other embodiments, the suture(s) can be heat set at, for example, 320° F. in individual units.

In other particular embodiments, the sutures 1008 can each comprise 12 filaments or “ends” of pin textured PET having a linear mass density of 1/20 Den/27 Fil. In some embodiments, the filaments can be braided together at a braid density of 10 PPI with alternating carrier tension to form the suture 1008. In other embodiments, the filaments can be braided with alternating carrier tension and variable pick density (e.g., variable PPI) to form the suture 1008. The suture(s) 1008 can be heat set at, for example, 320° F. while wrapped around a spool. In other embodiments, the suture(s) can be heat set at 320° F. in individual units.

In still other particular embodiments, the sutures 1008 can each comprise 12 filaments or “ends” of pin textured PET having a linear mass density of 1/20 Den/27 Fil. In some embodiments, the filaments can be braided together with alternating carrier tension and variable pick density (e.g., variable PPI) to form the suture. In other embodiments, the filaments can be braided together at a braid density of 10 PPI with alternating carrier tension to form the suture 1008. The suture(s) 1008 can be heat set at, for example, 320° F. while wrapped around a spool. In other embodiments, the suture(s) can be heat set at 320° F. in individual units.

In some embodiments, the sutures 1008 are assembled on the outer surface of the frame in the non-tensioned and expanded state (FIG. 33B). Once the prosthetic valve 1000 is implanted at a selected implantation site, the non-tensioned or relaxed sutures 1008 (FIG. 33B) can promote tissue ingrowth and improve PVL sealing. In some embodiments, the relaxed sutures 1008 can absorb and swell with blood. When in the relaxed configuration, as shown in FIG. 33B, yarns or filaments of the suture 1008 loosen from one another and fan radially outward from a longitudinal axis of the suture 1008 to create the fuzzy or puffy texture. The relaxed sutures 1008 can serve as a sealing member configured to prevent or mitigate PVL.

The sutures 1008 can be assembled on the frame such that selected portions of the suture 1008 are retained in the tensioned configuration and other portions are retained in the relaxed configuration. The tensioned and relaxed sections of the sutures 1008 can be retained in their tensioned or relaxed state by “locking” the ends of each tensioned or relaxed section to the frame for example, by knotting the suture 1008 to the frame, wrapping the suture 1008 around a junction or strut of the frame, and/or by using an additional suture to tie-off a portion of the suture 1008 in order to maintain certain portions of the suture 1008 in the relaxed or tensioned configurations. For example, in some embodiments, sections of the suture 1008 on the outer surface of the frame can be retained in a relaxed state to promote sealing, while sections of the suture 1008 on inside of the frame can be retained in a tensioned state to reduce crimp profile and/or for tightly securing other components (e.g., an inner skirt) to the frame. When forming a tensioned section, the suture 1008 can be locked (e.g., knotted or wrapped around a strut or junction) to the frame at a first location, pulled taught and then locked to the frame at a second location. When forming a relaxed section, the suture 1008 can be locked to the frame at first and second locations with the section of the suture between the first and second locations being in a non-tensioned relaxed state. A single suture 1008 can be used to form one or more tensioned sections and one or more relaxed sections at various locations on the prosthetic valve.

Though the illustrated embodiment of FIG. 32 shows four sutures 1008 configured as rings, it should be noted that in other embodiments any number of such sutures can be used. For example, in some embodiments, the prosthetic valve can comprise only a single expandable suture circumscribing an inflow end portion 1004 of the prosthetic valve 1000.

In alternative embodiments, a prosthetic valve can comprise a mechanically expandable valve, such as prosthetic valve 10, 100, or 400, having one or more sutures 1008 on the outside of the frame in lieu of or in addition to a sealing member on the outside of the frame.

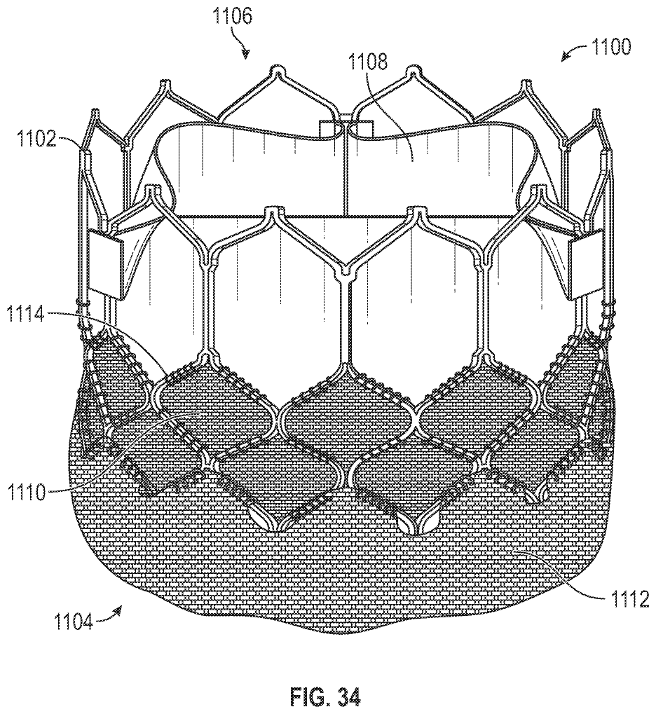

FIGS. 34-38 illustrate various embodiments of a prosthetic valve 1100. As shown in FIG. 34, the prosthetic valve 1100 can comprise frame 1102, inflow end portion 1104, outflow end portion 1106, and a valvular structure 1108. The prosthetic valve 1100 can further comprise an inner skirt 1110 and an outer skirt or sealing member 1112 coupled to an outer surface of the frame 1102. In the illustrated embodiment, as shown in FIG. 34, the inner skirt 1110 is coupled to an inner surface of the frame 1102. However, in other embodiments, the inner skirt 1110 can be disposed on the outer surface of the frame 1102 between the outer skirt 1112 and the frame 1102.

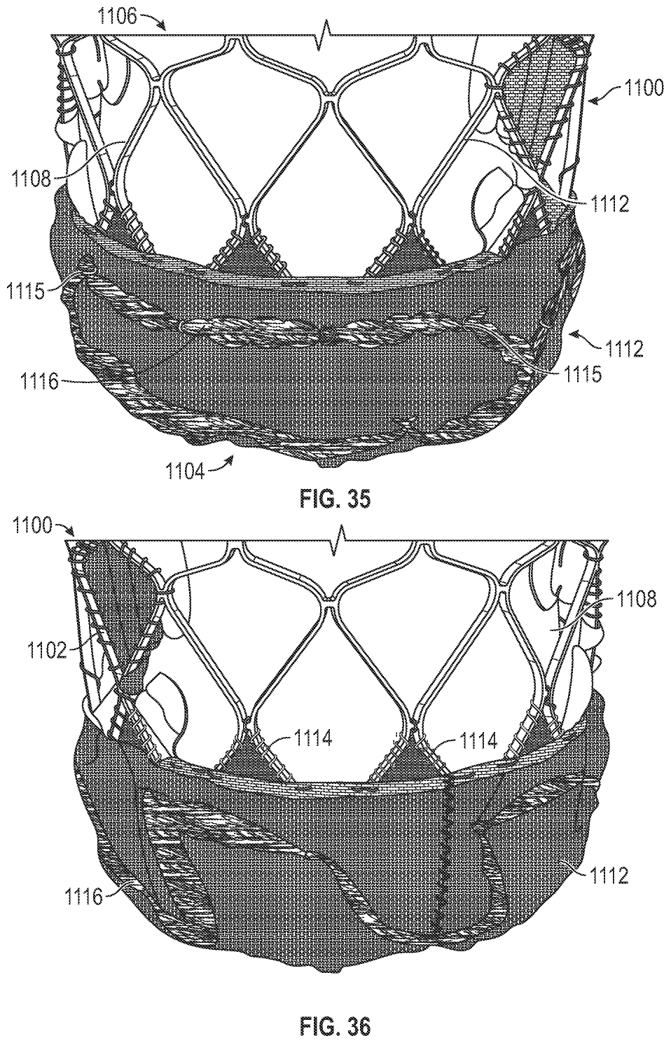

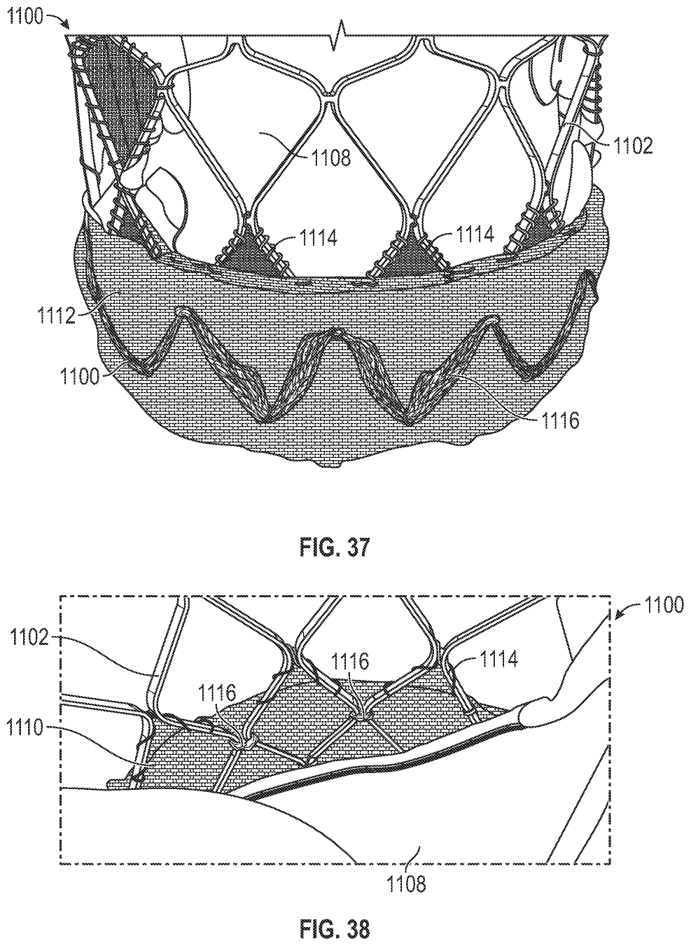

Referring now to FIGS. 35-37, in some embodiments, the outer skirt 1112 can comprise one or more expandable sutures 1116 (e.g., similar to expandable sutures 1008) sutured to an outer surface of the outer skirt 1112. In such embodiments, the sutures 1116 are configured to help the outer skirt 1112 serve as a sealing member to prevent or mitigate PVL. As shown in FIGS. 35-37, the suture(s) 1116 can be coupled to the outer skirt 1112 in any of various patterns selected to provide selected PVL sealing zones. For example, the sutures 1116 can be coupled to the outer skit in a sinusoidal or zig-zag pattern (see e.g., FIGS. 36-37), in a circular or ovular pattern (see e.g., FIG. 35), or any other pattern.

In some embodiments, as shown in FIGS. 35-37, the sutures 1116 are coupled to the outer skirt 1112 using one or more additional sutures 1115. However, in other embodiments, the sutures 1116 can be sewn through the outer skirt to couple the sutures 1116 to the skirt 1112. In such embodiments, the sutures 1116 can be tensioned (and therefore have a decreased diameter) at the locations where they are disposed on an inner surface of the skirt and/or frame to minimize the crimp profile of the prosthetic valve.

In some embodiments, the inner and/or outer skirts 1110, 1112 can be coupled to the frame 1102 using one or more sutures 1114. The sutures 1114 can extend along the edges of the inner and/or outer skirts 1110, 1112 to secure the skirts 1110, 1112 to the frame 1102. However, in some instances, the leaflets of the valvular structure can contact the sutures during systole, which can result in abrasion of the leaflets. To prevent or inhibit such abrasion, one or more expandable sutures 1116 (see e.g., FIG. 35) can be used in lieu of or in addition to the sutures 1114.

For example, as shown in FIG. 38, in some embodiments, the inner skirt 1110 can be coupled to the frame 1102 using one or more sutures 1114 and portions of one or more sutures 1116 can be stitched between junctions of the frame 1102 in a relaxed (i.e., non-tensioned or puffy) configuration. The relaxed sutures 1116 provide a soft, more absorbent structure (see e.g., 33B) that can absorb blood and minimize abrasion with the leaflets.

In other embodiments, the sutures 1116 can be used in lieu of sutures 1114 to couple the inner skirt 1110 to the frame. In such embodiments, the portions of the sutures 1116 disposed on the radially inner surface of the frame can be in the tensioned configuration and the portions disposed on the radially outer surface of the frame 1102 can be in the relaxed configuration. The tensioned and relaxed portions of each suture 1116 can be formed by locking the suture to locations on the frame, as previously described.

In still other embodiments, the sutures 1116 can be used to couple the inner skirt 1110 to the frame 1102 such that the sutures 1116 are predominantly disposed on the outer surface of the frame 1102. Such a configuration can further mitigate the risk of leaflet abrasion. The sutures 1116 can further be configured to promote tissue ingrowth around the prosthetic valve 1100, which can advantageously result in decreased PVL.

In some embodiments, rather than using the expandable sutures to secure one or more components of the prosthetic valve to each other, the expandable sutures 1116 can be used primarily to enhance sealing.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable prosthetic device, comprising:

a frame movable between a radially compressed and a radially expanded configuration, the frame comprising an inflow end portion, an outflow end portion, and a plurality of struts; and

a sealing member disposed around the frame, the sealing member comprising:

a cushioning layer comprising a plurality of texturized yarns extending along the longitudinal axis of the frame, and

a base layer disposed between the cushioning layer and the frame.

Example 2. The implantable prosthetic device of any example herein, particularly example 1, wherein the sealing member is resiliently stretchable between a first state corresponding to the radially expanded configuration of the frame, and a second state corresponding to the radially collapsed configuration of the frame.

Example 3. The implantable prosthetic device of any example herein, particularly any one of examples 1-2, wherein the base layer comprises a plain weave fabric including warp and weft yarns oriented perpendicularly to one another.

Example 4. The implantable prosthetic device of any example herein, particularly any one of examples 1-3, wherein an outflow end portion of the base layer comprises a plurality of projections corresponding to the shape of the struts.

Example 5. The implantable prosthetic device of any example herein, particularly any one of examples 1-4, wherein the cushioning layer comprises a first woven portion disposed at an outflow edge of the plurality of texturized yarns and a second woven portion disposed at an inflow edge of the plurality of texturized yarns.

Example 6. The implantable prosthetic device of any example herein, particularly any one of examples 1-5, wherein the plurality of texturized yarns is configured to float over a portion of the cushioning layer.

Example 7. The implantable prosthetic device of any example herein, particularly any one of examples 1-6, wherein an outflow end portion of the base layer is coupled to the frame and an inflow end portion of the cushioning layer is coupled to the frame.

Example 8. The implantable prosthetic device of any example herein, particularly any one of examples 1-6, wherein an inflow end portion of the cushioning layer extends past the base layer toward the inflow end of the frame.

Example 9. The implantable prosthetic device of any example herein, particularly example 8, wherein the inflow end portion of the cushioning layer extends over the inflow end portion of the frame.

Example 10. The implantable prosthetic device of any example herein, particularly any one of examples 1-6, wherein an inflow edge of the cushioning layer is coupled to the frame at a first location, wherein an outflow edge of the cushioning layer is coupled to the frame at a second location, and wherein the base layer is not coupled to the frame.

Example 11. The implantable prosthetic device of any example herein, particularly any one of examples 1-10, wherein the base layer comprises a first set of yarns extending in a first direction and a second set of yarns extending in a second direction, and wherein the first and second yarns are oriented at a non-perpendicular angle relative to one another.

Example 12. The implantable prosthetic device of any example herein, particularly example 11, wherein the non-perpendicular angle is a 45 degree angle.

Example 13. The implantable prosthetic device of any example herein, particularly any one of examples 1-12, wherein the base layer comprises a first set of yarns extending in a first direction and a second set of yarns extending in a second direction, and wherein the first and second yarns are oriented at a non-perpendicular angle relative to an inflow edge of the base layer.

Example 14. The implantable prosthetic device of any example herein, particularly example 13, wherein the non-perpendicular angle is a 45 degree angle.

Example 15. The implantable prosthetic device of any example herein, particularly any one of example 1-14, wherein the cushioning layer further comprises a plurality of elastic yarns extending along the longitudinal axis of the frame and interspersed with the texturized yarns.

Example 16. The implantable prosthetic device of any example herein, particularly any one of examples 1-15, wherein the cushioning layer comprises a leno weave portion extending circumferentially around at least a portion of the cushioning layer.

Example 17. The implantable prosthetic device of any example herein, particularly example 16, wherein the leno weave portion comprises first and second leno yarns configured to trap one or more texturized yarns between them.

Example 18. The implantable prosthetic device of any example herein, particularly any one of examples 1-17, wherein the sealing member has a first edge and a second edge, and wherein the first and second edges are coupled together such that the sealing member forms a cylinder.

Example 19. The implantable prosthetic device of any example herein, particularly example 18, wherein the sealing member comprises one or more circumferentially extending yarns each having a first extending portion and a second extending portion, and wherein the first and second extending portions can be tied together to couple the first and second circumferential edges of the sealing member.

Example 20. The implantable prosthetic device of any example herein, particularly example 18, wherein the first and second edges can be coupled together via a chain stitch.

Example 21. The implantable prosthetic device of any example herein, particularly example 20, wherein the chain stitch is disposed in a sinusoidal pattern.

Example 22. The implantable prosthetic device of any example herein, particularly any one of examples 20-21, wherein the chain stitch passes one or more times over a joint where the first and second circumferential edges abut.

Example 23. An implantable prosthetic device, comprising:

a frame movable between a radially compressed and a radially expanded configuration, the frame comprising an inflow end portion, an outflow end portion, and a plurality of struts; and

a sealing member circumscribing the frame, the sealing member comprising
a first layer disposed radially outwardly of the frame, the first layer configured to promote radially outward thrombus formation between the implantable prosthetic device and a selected implantation site, and
a second layer disposed between the first layer and the frame, the second layer configured to inhibit radially inward thrombus formation.

Example 24. The implantable prosthetic device of any example herein, particularly example 23, wherein the first layer comprises a plurality of texturized yarns disposed in a direction parallel to a longitudinal axis of the frame.

Example 25. The implantable prosthetic device of any example herein, particularly any one of examples 23-24, wherein the sealing member is resiliently stretchable between a first state corresponding to the radially expanded configuration of the frame, and a second state corresponding to the radially collapsed configuration of the frame.

Example 26. The implantable prosthetic device of any example herein, particularly any one of examples 23-25, wherein the second layer comprises a plain weave fabric including warp and weft yarns oriented perpendicularly to one another.

Example 27. The implantable prosthetic device of any example herein, particularly any one of examples 23-26, wherein an outflow end portion of the second layer comprises a plurality of projections corresponding to the shape of the struts.

Example 28. The implantable prosthetic device of any example herein, particularly any one of examples 23-26, wherein an outflow end portion of the second layer is coupled to the frame and an inflow end portion of the first layer is coupled to the frame.

Example 29. The implantable prosthetic device of any example herein, particularly any one of examples 23-26, wherein an inflow end portion of the first layer extends past the second layer toward the inflow end of the frame.

Example 30. The implantable prosthetic device of any example herein, particularly example 29, wherein the inflow end portion of the first layer extends over the inflow end portion of the frame.

Example 31. The implantable prosthetic device of any example herein, particularly any one of examples 23-26, wherein an inflow edge of the first layer is coupled to the frame at a first location, wherein an outflow edge of the first layer is coupled to the frame at a second location, and wherein the second layer is not coupled to the frame.

Example 32. The implantable prosthetic device of any example herein, particularly any one of examples 23-31, wherein the second layer comprises a first set of yarns extending in a first direction and a second set of yarns extending in a second direction, and wherein the first and second yarns are oriented at a non-perpendicular angle relative to one another.

Example 33. The implantable prosthetic device of any example herein, particularly example 32, wherein the non-perpendicular angle is a 45 degree angle.

Example 34. The implantable prosthetic device of any example herein, particularly any one of examples 23-33, wherein the second layer comprises a first set of yarns extending in a first direction and a second set of yarns extending in a second direction, and wherein the first and second yarns are oriented at a non-perpendicular angle relative to an inflow edge of the second layer.

Example 35. The implantable prosthetic device of any example herein, particularly example 34, wherein the non-perpendicular angle is a 45 degree angle.

Example 36. The implantable prosthetic device of any example herein, particularly any one of examples 23-35, wherein the first layer further comprises a plurality of elastic yarns extending along the longitudinal axis of the frame and interspersed with the texturized yarns.

Example 37. The implantable prosthetic device of any example herein, particularly any one of examples 23-36, wherein the second layer is treated with one or more agents configured to inhibit thrombus formation.

Example 38. The implantable prosthetic device of any example herein, particularly any one of examples 23-37, wherein the first layer comprises a leno weave portion extending circumferentially around at least a portion of the first layer.

Example 39. The implantable prosthetic device of any example herein, particularly example 38, wherein the leno weave portion comprises first and second leno yarns configured to trap one or more texturized yarns between them.

Example 40. The implantable prosthetic device of any of any example herein, particularly any one of examples 23-39, wherein the sealing member has a first edge and a second edge, and wherein the first and second edges are coupled together such that the sealing member forms a cylinder.

Example 41. The implantable prosthetic device of any example herein, particularly example 40, wherein the sealing member comprises one or more circumferentially extending yarns each having a first extending portion and a second extending portion, and wherein the first and second extending portions can be tied together to couple the first and second edges of the sealing member to one another.

Example 42. The implantable prosthetic device of any example herein, particularly any one of example 40, wherein the first and second edges can be coupled together via a chain stitch.

Example 43. The implantable prosthetic device of any example herein, particularly example 42, wherein the chain stitch is disposed in a sinusoidal pattern.

Example 44. The implantable prosthetic device of any example herein, particularly any one of examples 42-43, wherein the chain stitch passes one or more times over a joint where the first and second circumferential edges abut.

Example 45. The implantable prosthetic device of any example herein, particularly any one of examples 23-45, wherein the first layer comprises one or more expandable sutures.

Example 46. The implantable prosthetic device of any example herein, particularly example 45, wherein the expandable sutures are coupled to the second layer in a sinusoidal pattern.

Example 47. A method of making an angled weave fabric, comprising:

disposing a set of first yarns on a loom such that the first yarns extend in a first direction;

weaving a shuttle coupled to a second yarn through the set of first yarns in a second direction such that a portion of the second yarn is oriented perpendicularly relative to the set of first yarns;

moving the portion of the second yarn against an angled base member such that the portion is oriented at a non-perpendicular angle relative to the set of first yarns.

Example 48. The method of any example herein, particularly example 47, wherein the non-perpendicular angle is a 45 degree angle.

Example 49. The method of any example herein, particularly any one of examples 47-48, further comprising:

weaving the shuttle coupled to the second yarn through the set of first yarns in the second direction such that an additional portion of the second yarn is oriented perpendicularly relative to the set of first yarns;

moving the additional portion against the angled base member such that the additional portion is oriented at a non-perpendicular angle relative to the set of first yarns.

Example 50. The method any example herein, particularly example 49, further comprising repeating as necessary until a selected width of second yarn portions is reached.

Example 51. A method of making an implantable prosthetic device, comprising:

expanding a mechanically-expandable frame comprising a first set of struts and a second set of struts, wherein each strut of the first set of struts is coupled to one or more struts of the second set of struts, to a non-working diameter wherein the first set of struts are oriented perpendicularly with respect to the second set of struts;

disposing a sealing member comprising a plurality of warp and weft yarns over the frame such that the warp yarns are aligned with the first set of struts and the weft yarns are aligned with the second set of struts; and coupling the sealing member to the frame.

Example 52. The method of any example herein, particularly example 51, wherein the non-working diameter is 23 mm.

Example 53. The method of any example herein, particularly any one of examples 51-52, further comprising compressing the mechanically-expandable frame to a compressed diameter such that the sealing member elongates axially.

Example 54. The method of any example herein, particularly any one of examples 51-53, wherein disposing the sealing member over the frame comprises aligning one or more markings on the sealing member with the struts of the frame, the markings configured to show the directions of the warp and weft yarns.

Example 56. The method of any example herein, particularly any one of examples 51-54, wherein coupling the sealing member to the frame comprises suturing the sealing member to the frame at a first location using a suture, the suture extending through a first aperture in the sealing member, around a respective strut, and back through the first aperture.

Example 57. The method of any example herein, particularly example 56, further comprising extending a portion of the suture along a geometric centerline of the respective strut and suturing the sealing member to the frame at a second location, the suture extending through a second aperture in the sealing member, around the respective strut, and back through the second aperture.

Example 58. An implantable prosthetic device, comprising:

a frame movable between a radially compressed and a radially expanded configuration, the frame comprising an inflow end portion, an outflow end portion, and a plurality of struts defining a plurality of cells; and a sealing member comprising at least one expandable suture configured to move between a tensioned configuration and a relaxed configuration when immersed in blood.

Example 59. The implantable prosthetic device of any example herein, particularly example 58, wherein the sealing member circumscribes the frame.

Example 60. The implantable prosthetic device of any example herein, particularly any one of examples 58-59, wherein the at least one expandable suture comprises a plurality of expandable sutures, and wherein the plurality of expandable sutures comprises one or more horizontal sutures disposed around a circumference of the frame and one or more vertical sutures extending between opposing junctions of a respective cell.

Example 61. The implantable prosthetic device of any example herein, particularly any one of examples 58-60, wherein the sealing member further comprises a base layer and wherein the at least one expandable suture is coupled to the base layer.

Example 62. An implantable prosthetic device, comprising:

a frame movable between a radially compressed and a radially expanded configuration, the frame comprising an inflow end portion, an outflow end portion, and a plurality of struts defining a plurality of cells; and

a sealing member comprising at least one expandable suture, the expandable suture configured to move between a relaxed first configuration and a narrower second configuration when the expandable suture is placed in tension.

Example 63. The implantable prosthetic device of any example herein, particularly example 62, wherein portions of the expandable suture are disposed on the frame in the first configuration and portions of the expandable suture are disposed on the frame in the second configuration.

Example 64. The implantable prosthetic device of any example herein, particularly any one of examples 62-63, wherein the expandable suture comprises twelve filaments.

Example 65. The implantable prosthetic device of any example herein, particularly any one of examples 62-64, wherein the expandable suture comprises polyester.

Example 66. The implantable prosthetic device of any example herein, particularly any one of examples 62-65, wherein the sealing member further comprises an outer skirt on which the expandable suture is at least partially disposed.

In view of the many possible embodiments to which the principles of the disclosure may be applied, it should be recognized that the illustrated embodiments are only preferred examples and should not be taken as limiting the scope. Rather, the scope is defined by the following claims. We therefore claim all that comes within the scope and spirit of these claims.

We claim:

1. An implantable prosthetic device, comprising:
a frame movable between a radially compressed and a radially expanded configuration, the frame comprising an inflow end portion, an outflow end portion, and a plurality of struts; and
a sealing member disposed around the frame, the sealing member comprising:
a cushioning layer comprising a plurality of texturized yarns extending along the longitudinal axis of the frame, and
a base layer disposed between the cushioning layer and the frame.

2. The implantable prosthetic device of claim 1, wherein the sealing member is resiliently stretchable between a first state corresponding to the radially expanded configuration of the frame, and a second state corresponding to the radially collapsed configuration of the frame.

3. The implantable prosthetic device of claim 1, wherein the base layer comprises a plain weave fabric including warp and weft yarns oriented perpendicularly to one another.

4. The implantable prosthetic device of claim 1, wherein an outflow end portion of the base layer comprises a plurality of projections corresponding to the shape of the struts.

5. The implantable prosthetic device of claim 1, wherein the cushioning layer comprises a first woven portion disposed at an outflow edge of the plurality of texturized yarns and a second woven portion disposed at an inflow edge of the plurality of texturized yarns.

6. The implantable prosthetic device of claim 1, wherein the plurality of texturized yarns is configured to float over a portion of the cushioning layer.

7. The implantable prosthetic device of claim 1, wherein an outflow end portion of the base layer is coupled to the frame and an inflow end portion of the cushioning layer is coupled to the frame.

8. The implantable prosthetic device of claim 1, wherein an inflow end portion of the cushioning layer extends past the base layer toward the inflow end of the frame.

9. The implantable prosthetic device of claim 8, wherein the inflow end portion of the cushioning layer extends over the inflow end portion of the frame.

10. The implantable prosthetic device of claim 1, wherein an inflow edge of the cushioning layer is coupled to the frame at a first location, wherein an outflow edge of the cushioning layer is coupled to the frame at a second location, and wherein the base layer is not coupled to the frame.

11. The implantable prosthetic device of claim 1, wherein the base layer comprises a first set of yarns extending in a first direction and a second set of yarns extending in a second direction, and wherein the first and second yarns are oriented at a non-perpendicular angle relative to one another.

12. The implantable prosthetic device of claim 11, wherein the non-perpendicular angle is a 45 degree angle.

13. The implantable prosthetic device of claim 1, wherein the base layer comprises a first set of yarns extending in a first direction and a second set of yarns extending in a second direction, and wherein the first and second yarns are oriented at a non-perpendicular angle relative to an inflow edge of the base layer.

14. The implantable prosthetic device of claim 13, wherein the non-perpendicular angle is a 45 degree angle.

15. The implantable prosthetic device of claim 1, wherein the cushioning layer further comprises a plurality of elastic yarns extending along the longitudinal axis of the frame and interspersed with the texturized yarns.

16. The implantable prosthetic device of claim 1, wherein the cushioning layer comprises a leno weave portion extending circumferentially around at least a portion of the cushioning layer.

17. The implantable prosthetic device of claim 16, wherein the leno weave portion comprises first and second leno yarns configured to trap one or more texturized yarns between them.

18. The implantable prosthetic device of claim 1, wherein the sealing member has a first edge and a second edge, and wherein the first and second edges are coupled together such that the sealing member forms a cylinder.

19. The implantable prosthetic device of claim 18, wherein the sealing member comprises one or more circumferentially extending yarns each having a first extending portion and a second extending portion, and wherein the first and second extending portions can be tied together to couple the first and second edges of the sealing member.

20. The implantable prosthetic device of claim 18, wherein the first and second edges can be coupled together via a chain stitch.

21. The implantable prosthetic device of claim 20, wherein the chain stitch is disposed in a sinusoidal pattern.

22. The implantable prosthetic device of claim 20, wherein the chain stitch passes one or more times over a joint where the first and second edges abut.
